(19) United States
(12) Patent Application Publication
(10) Pub. No.: US 2015/0286773 A1
(43) Pub. Date: Oct. 8, 2015

(54) METHODS AND SYSTEMS FOR DIAGNOSING PRENATAL ABNORMALITIES

(71) Applicant: THE GENERAL HOSPITAL CORPORATION, Boston, MA (US)

(72) Inventors: Michael Talkowski, Swampscott, MA (US); James F. Gusella, Boston, MA (US); Cynthia Morton, Boston, MA (US)

(21) Appl. No.: 14/443,307

(22) PCT Filed: Nov. 15, 2013
(86) PCT No.: PCT/US2013/070429

§ 371 Date: May 15, 2015

Related U.S. Application Data
(60) Provisional application No. 61/726,923, filed on Nov. 15, 2012.

(51) Int. Cl. G06F 19/18; C12Q 1/68

(52) U.S. Cl. CPC G06F 19/18; C12Q 1/6883; C12Q 2600/16; C12Q 2600/118

(57) ABSTRACT

Embodiments of various aspects described herein are directed to methods and systems for performing a prenatal diagnostic testing. In some embodiments, the methods and systems described herein can be used to diagnose structural rearrangements or chromosome breakpoints in a prenatal sample. In some embodiments, the methods and systems described herein can be used to diagnose de novo balanced chromosomal rearrangements in a prenatal sample.

### CROSS REFERENCE TO RELATED APPLICATIONS

This application claims the benefit under 35 U.S.C. §119(e) of U.S. Provisional Application No. 61/726,923 filed Nov. 15, 2012, the contents of which are incorporated herein by reference in its entirety.

### GOVERNMENT SUPPORT

This invention was made with government support under grant nos. HD065286, GM061354, and MH087123 all awarded by the National Institutes of Health (NIH). The government has certain rights in the invention.

### TECHNICAL FIELD OF THE DISCLOSURE

The inventions described herein generally relate to methods and systems for performing a genetic diagnostic testing. In some embodiments, the methods and systems described herein can be used to diagnose structural rearrangements or chromosome breakpoints in a sample. In some embodiments, the methods and systems described herein can be used to diagnose de novo balanced chromosomal rearrangements in a sample.

### BACKGROUND

Deep sequencing of the whole genome holds diagnostic promise but is currently believed to be impractical for routine prenatal care. The risk of major structural birth defects among live births in the United States is approximately 3% [1] and is associated with inherited or de novo genetic rearrangements and mutations as well as with maternal factors, such as advanced age, certain clinical conditions, and exposure to teratogenic factors. Approximately 1 in 2000 prenatal cases analyzed with conventional karyotyping has a de novo, apparently balanced reciprocal translocation that carries a 6.1% risk of congenital malformation [2]. Ultrasound examination between 18 and 20 weeks of gestation allows detection of major malformations and is offered routinely, since 90% of infants with congenital anomalies are born to women without predisposing risk factors [3]. An abnormal finding on fetal ultrasonography generally necessitates counseling and a discussion of a diagnostic procedure that can be used to assess the possibility that the abnormality has a genetic basis.

Conventional karyotyping, which is the standard method used for prenatal cytogenetic diagnosis, can detect numerical abnormalities as well as unbalanced and apparently balanced rearrangements within microscopical resolution (range, ˜3 to ˜10 Mb). Fluorescence in situ hybridization analyses can be used to detect chromosomal abnormalities smaller than 3 Mb, but this method is not suitable for high-throughput analyses because only a limited number of probes can be screened simultaneously. Array-based comparative genomic hybridization (CGH) has been introduced in prenatal diagnosis to detect genomewide gains and losses with higher resolution [4] but its use is typically limited to dosage imbalances on the order of tens to hundreds of kilobases. For example, a recent study of more than 36,000 persons revealed karyotypic abnormalities in 0.78% of persons with intellectual disabilities in whom array-based CGH tests were unremarkable [5]. Accordingly, there is a need to rapidly localize breakpoints of cytogenetically balanced chromosomal rearrangements to individual genes, which can substantially improve the prediction of phenotypic outcomes and inform postnatal medical care.

### SUMMARY

Conventional cytogenetic testing offers low-resolution detection of balanced karyotypic abnormalities but cannot provide the precise, gene-level knowledge required to predict outcomes. The use of high-resolution whole-genome deep sequencing is currently impractical for the purpose of routine clinical care. To this end, the inventors have developed, inter alia, a novel approach of whole-genome analyses that can be used for nucleotide-level genetic diagnostics within a time frame that is more practical for use in clinical actions. Specifically, the inventors have demonstrated that large-insert sequencing of DNA extracted from amniotic-fluid cells of a patient can detect the presence of a balanced de novo translocation. The inventors have used massively parallel paired-end sequencing of customized large-insert jumping libraries to define the precise consequences of a balanced de novo translocation in DNA extracted from amniotic-fluid cells, and thus to determine chromosomal breakpoints. In one embodiment, the inventors have used the methods described herein to determine direct disruption of CHD7, a causal locus in the CHARGE syndrome (coloboma of the eye, heart anomaly, atresia of the choanae, retardation, and genital and ear anomalies), illustrating the applications and advantages of the methods described herein in prenatal diagnosis, when used alone or in combination with prenatal karyotyping. The methods described herein are generalizable to any analysis of genomic DNA currently studied, for example, by karyotyping, providing data that are substantially more detailed, with resolution that can be as precise as identifying the exact nucleotide breakpoints for a range of different genetic rearrangements or anomalies.

One aspect provided herein relates to a method of prenatal determination of chromosomal abnormalities. The method comprises (a) subjecting genomic DNA extracted from cells in an amniotic fluid sample to whole-genome sequence analysis using a large-insert jumping library; and (b) identifying, using a specifically-programmed computer system, structural rearrangements or chromosomal breakpoints in the DNA based on the sequencing data, thereby detecting the presence of one or more abnormalities in the genomic DNA of a fetus associated with structural rearrangements or chromosomal breakpoints.

Another aspect provided herein relates to a method of postnatal determination of chromosomal abnormalities. The method comprises (a) subjecting genomic DNA extracted from tissue to whole-genome sequence analysis using a large-insert jumping library; and (b) identifying, using a specifically-programmed computer system, structural rearrangements or chromosomal breakpoints in the DNA based on the sequencing data, thereby detecting the presence of one or more abnormalities in the genomic DNA of a human subject associated with structural rearrangements or chromosomal breakpoints.

In some embodiments of various aspects described herein, the large-insert jumping library can created by a process comprising: (a) size-selecting fragments of the genomic DNA; (b) circularizing the size-selected DNA fragments with adaptors comprising a first member of an affinity binding pair and an optional endonuclease recognition site; (c) fragmenting the circularized DNA into linear DNA fragments in the presence of an endonuclease specific for the endonuclease recognition site present in the adaptors or by random shearing the circularized DNA, wherein at least a portion of the linear DNA fragments comprise the adaptors of step (b) and an end sequence derived from the genomic DNA on each end of the linear DNA fragments; (d) contacting the linear DNA fragments with a solid support comprising a second member of the affinity binding pair, thereby selecting linear DNA fragments comprising the first member of the affinity binding pair and the end sequence on either end; (e) amplifying the linear DNA fragments bound to the solid support, thereby generating a library of DNA fragments comprising end sequences derived from the genomic DNA, wherein the end sequences are separated by a genomic distance equal to the size of the size-selected DNA fragments.

In some embodiments, the specifically-programmed computer system can comprise one or more processors; and memory to store one or more programs, the one or more programs comprising instructions for: (a) aligning paired-end sequence reads obtained from the whole-genome sequence analysis against sequence of at least one or more chromosomes; (b) categorizing as anomalous, those read pairs that align to genomic sequences separated by significantly greater than or less than the size of the DNA fragments selected for library creation, that have unexpected orientations, or for which the corresponding end sequences align to different chromosomes; (c) categorizing the anomalous read pairs into the same cluster if both sides of the read pairs align within a selected distance of each other; wherein each output cluster represents a putative structural variant breakpoint; and (d) displaying a content that comprises a signal indicative of information associated with the output clusters, wherein the signal is selected from the group consisting of a signal indicative of one or more detectable structural variant breakpoints; a signal indicative of no detectable structural variant breakpoints; a signal indicative of a normal sample, a signal indicative of a disease or disorder associated with the detectable structural variant breakpoints, and any combination thereof.

Systems, e.g., for the analysis of whole genome sequence data, are also provided herein. The system comprises: (a) a determination module configured to receive said at least one test sample and perform at least one sequencing analysis on said at least one test sample; (b) a storage device configured to store output sequence data from said determination module; (c) a computing module comprising specifically-programmed instructions to determine from the output sequence data validity or invalidity of structural rearrangements represented by the output sequence data, wherein the instructions comprise: (i) mapping read-pairs of the output sequence data against a reference genome; (ii) categorizing the read-pairs into clusters based on at least one common feature; (iii) removing the read-pair clusters having their mapping positions localized to predefined centromeric, telomeric, or heterochromatic regions over the reference genome; (iv) measuring at least two or more features of the remaining read-pair clusters, wherein said features are selected from the group consisting of: number of read-pairs in the cluster; mapping quality scores on both ends of the cluster and the residual between both measurements; read-pair uniqueness across both ends of the cluster and the residual between both measurements; distance between the maximum and minimum mapping positions on both ends of the cluster; normalized distance between the maximum and minimum mapping positions on both ends of the cluster; local coverage ratio of the number of the read-pairs in the cluster to the number of proper pairs at a breakpoint junction in the cluster; global coverage ratio of the number of the read-pairs in the cluster to average haploid proper pair coverage in the reference genome; GC percent averaging across regions on both ends of the cluster and the residual between both measurements; alignability percent averaging across sequences of both ends of the cluster and the residual between both measurements; and any combinations thereof; (v) performing a pre-trained decision tree classification program to determine the validity or invalidity of the structural rearrangements represented by the clusters; and (d) a display module for displaying a content based in part on the data output from said computing module, wherein the content comprises a signal indicative of the presence of valid structural rearrangements, or a signal indicative of the absence of any valid structural rearrangements.

### BRIEF DESCRIPTION OF THE DRAWINGS

FIGS. 1A-1F show clinical findings detected with prenatal imaging. A transaxial ultrasonogram with a four-chamber view of the heart, obtained at 27.3 weeks of gestation (FIG. 1A), shows a small right ventricle (arrow), as compared with the left ventricle (star), which was first detected at 18.8 weeks; tricuspid atresia was also detected on earlier imaging. A transaxial ultrasonogram obtained at 35.3 weeks of gestation (FIG. 1B) shows polyhydramnios (dashed line), first detected at 30.4 weeks; also noteworthy is the absence of a fluid-filled stomach in the upper abdomen (arrow). An ultrasonogram of the fetal profile (FIG. 1C) and a three-dimensional ultrasonogram of the fetal face (FIG. 1D), both obtained at 34.4 weeks of gestation, show microstomia and protrusion of the upper lip (FIG. 1D, arrow), and a three-dimensional ultrasonogram obtained at 33.3 weeks of gestation (FIG. 1E) shows abnormally clenched hands and flexed arms. A transaxial ultrasonogram of the perineum in a phenotypic male fetus, obtained at 35.3 weeks of gestation, shows only one testicle in the scrotum (FIG. 1F, arrow).

FIGS. 2A-2B show karyotype analysis of amniotic-fluid cells. FIGS. 2A and 2B show complete and partial (chromosomes 6 and 8, derivative chromosomes on the right of each chromosome pair) karyograms of cultured 33.3 weeks amniotic-fluid cells reported as 46,XY,t(6; 8)(q13; q13)dn, respectively. The karyotype was subsequently revised following DNA sequencing to 46,XY,t(6; 8)(q13; q12.2)dn with next-generation cytogenetics nomenclature of 46,XY,t(6; 8)(6pter->70405867::chr8 61628671->qter; 8pter->61628669::chr6 70405868->qter).

FIG. 3 is an exemplary sequencing and analysis timeline. The delineation of a de novo balanced translocation initially reported as 46,XY,t(6; 8)(q13; q13)dn is revised, after DNA sequencing, to 46,XY,t(6; 8)(q13; q12.2)dn. In step 1A, 2-kb jumping libraries are prepared from genomic DNA, and in step 1B, the final distribution of fragment sizes is shown. In step 2, massively parallel paired-end 25-cycle sequencing of DNA fragments is performed on an Illumina HiSeq 2000. In step 3, computational analyses are performed, including distributed parallel alignment of sequenced reads and clustering of anomalous read pairs (step 3A) and identification of candidate translocation clusters (step 3B). The inset in step 3B shows an example of a theoretical distribution of reads spanning a translocation breakpoint on a derivative chromosome. In step 4, the translocation breakpoint is confirmed by means of a polymerase-chain-reaction assay, and Sanger sequencing informs the precise breakpoint in the initial karyotype. In the panel for step 4, the breakpoint on der(8) is delineated on the Sanger sequencing reads. Chromatogram peaks are shown at the top, and a nucleotide sequence from a fragment crossing the breakpoint is shown in the bottom of the panel for step 4.

FIG. 4 shows a flow diagram of exemplary cluster identification and filtering. The analysis pipeline identified 21,564 initial chimeric clusters of more than three read pairs of any quality. Filtering required a minimum mapping quality of Q20, more than eight read pairs per candidate cluster (>25% of expected coverage based on the alignment metrics) but less than double the expected coverage, and a minimum cluster uniqueness of 90% (i.e., more than 90% of the read pairs within the cluster required unique mapping positions). It was then filtered for common variants observed in more than 25% of individuals sequenced as previously reported in Chiang et al. Nat Genet 2012; 44:390-397, representing either human genome reference artifacts or common standing variation. This resulted in seven candidate clusters genome-wide, but only one cluster that localized to chromosomes 6 and 8.

FIG. 5 is a 3-dimensional plot showing chimeric clusters between chromosomes 6 and 8. These data illustrate the clear distinction between the true ‘diagnostic cluster’ (i.e. cluster of read-pairs delineating the karyotypically identified event) on chromosomes 6 and 8 from false positive chimeric clusters between these chromosomes. Map quality is represented on the Y-axis from 0 (not unique) to 35, and cluster size (number of read pairs within a cluster) is represented on the X-axis. The Z-axis defines the frequency of clusters in each bin. As displayed, the true translocation event was the only chimeric pair that met the criteria of a candidate cluster between these two chromosomes.

FIG. 6 shows sequence-based delineation of a balanced de novo translocation. Sequencing revealed a balanced translocation disrupting CHD7 at 8q12.2 and disrupting LMBRD1 at 6q13. CHD7 and LMBRD1 are transcribed on opposite strands in the translocation and are incompatible with the formation of a fusion transcript. Normal chromosomes 6 and 8 are shown, as are the derivative chromosomes, after translocation. The breakpoint region is expanded in the middle, showing the cytogenetic band, the genomic coordinates of each chromosome, the precise breakpoint (dashed lines) on each derivative, and the nucleotide sequence of the junction point, including microhomology (boxed) at the breakpoint.

FIG. 7 is graphical presentation of genomic localization of CHD7 in comparison to karyotypically reported breakpoint at 8q13. The definition of 8q13 as the cytogenetic breakpoint would not have implicated CHD7 at 8q12.2. Left: Nucleotide locations of the 5′ end of CHD7 in 8q12.2 and the middle of the karyotypically reported breakpoint band at 8q138 with the total number of base pairs and genes located between these loci (NCBI Map Viewer, Build 37.3). Right: Karyotypically visible bands adjacent to 8q13 (8q12 and 8q21) would entail at least 38 Mb from the proximal point of 8q12 (at 8q12.1) to the distal point of 8q21 (at 8q21.3). There are 288 potential phenotype contributing loci in this region (NCBI Map Viewer, Build 37.3), including 39 disease related loci, as well as three other syndromic loci in addition to CHD7 that involve cardiac anomalies (OMIM Gene Map).

FIG. 8 is a block diagram showing an exemplary system that can be for use in the methods described herein, e.g., for prenatal or postnatal determination of chromosomal abnormalities.

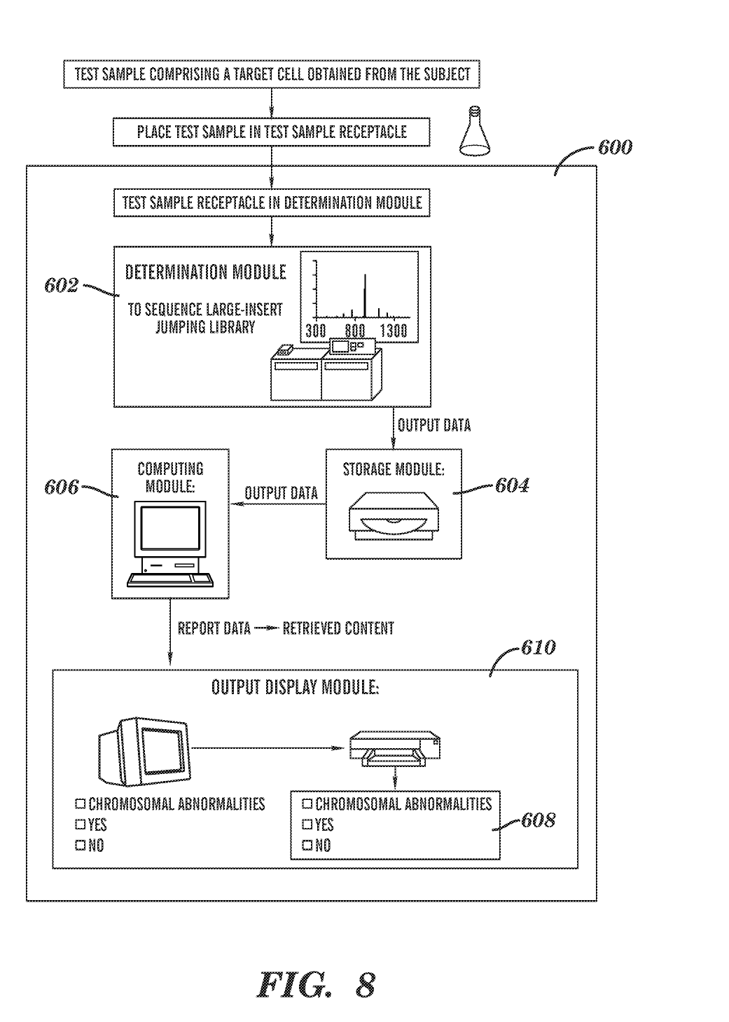

FIG. 9 is an exemplary set of instructions on a computer readable storage medium for use with the systems described herein.

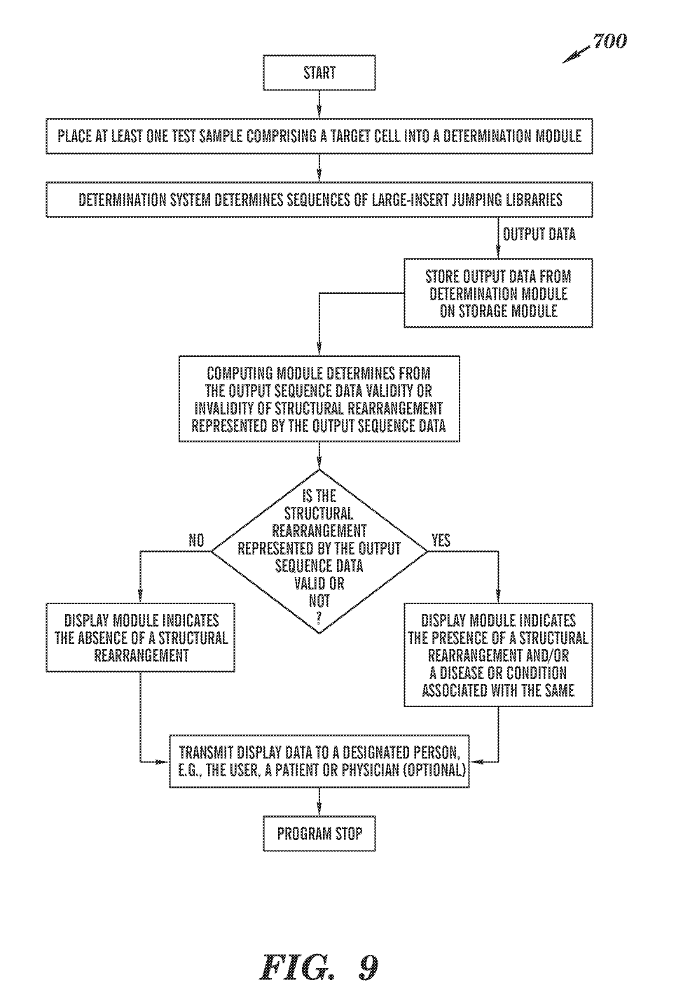

### DETAILED DESCRIPTION OF THE INVENTION

Conventional cytogenetic testing offers low-resolution detection of balanced karyotypic abnormalities but cannot provide the precise, gene-level knowledge required to predict outcomes. The use of high-resolution whole-genome deep sequencing is currently impractical for the purpose of routine clinical care. To this end, the inventors have developed, inter alia, a novel approach of whole-genome analyses that can be used for nucleotide-level genetic diagnostics within a time frame that is more practical for use in clinical actions. Specifically, as proof of principle, the inventors have applied the method, which is applicable to any genomic analysis, to prenatal diagnosis, demonstrating that large-insert sequencing of DNA extracted from amniotic-fluid cells of a patient can detect the presence of a balanced de novo translocation. The inventors have used massively parallel paired-end sequencing of customized large-insert jumping libraries to define the precise consequences of a balanced de novo translocation in DNA extracted from amniotic-fluid cells, and thus to determine chromosomal breakpoints. In one embodiment, the inventors have used the methods described herein to determine direct disruption of CHD7, a causal locus in the CHARGE syndrome (coloboma of the eye, heart anomaly, atresia of the choanae, retardation, and genital and ear anomalies), illustrating the applications and advantages of the methods described herein in prenatal and postnatal diagnosis, when used alone or in combination with prenatal karyotyping. Accordingly, embodiments of various aspects relate to methods and/or systems for use in prenatal or postnatal diagnosis.

As used herein, the term “prenatal diagnosis” refers to the determination of the health and conditions of a fetus, including detection of defects or abnormalities as well as the diagnosis of diseases. A variety of non-invasive and invasive techniques are currently available for prenatal diagnosis. These techniques include, for example, ultrasonography, maternal serum screening, amniocentesis, and chorionic villus sampling (or CVS). The terms “sonographic examination”, “ultrasonographic examination”, and “ultrasound examination” are used herein interchangeably. They refer to a clinical non-invasive procedure in which high frequency sound waves are used to produce visible images from the pattern of echos made by different tissues and organs of the fetus. A sonographic examination may be used to determine the size and position of the fetus, the size and position of the placenta, the amount of amniotic fluid, and the appearance of fetal anatomy. Ultrasound examinations can reveal the presence of congenital anomalies (i.e., anatomical or structural malformations that are present at birth).

As used herein, the term “postnatal diagnosis” refers to determination of the health and condition of a human subject, including detection of genetic defects or abnormalities as well as the diagnosis of diseases. In some embodiments, the term “postnatal diagnosis” refers to determination of the health and conditions of a newborn, including detection of genetic defects or abnormalities as well as the diagnosis of diseases.

The term “amniocentesis”, as used herein, refers to a prenatal test performed by inserting a long needle in the mother's lower abdomen into the amniotic cavity inside the uterus using ultrasound to guide the needle, and withdrawing a small amount of amniotic fluid. The amniotic fluid contains cells from the fetus and/or cell-free fetal DNA.

However, the existing prenatal diagnostic methods cannot detect de novo balanced chromosomal rearrangements. A balanced rearrangement generally refers to the relocation of genomic material between different chromosomes or within the same chromosome without a loss or gain in the genomic material. The term “de novo” as used herein is used for abnormalities that are newly detected in a fetus and not inherited from one of the parents. The methods of prenatal diagnosis as described herein can be used to determine chromosomal abnormalities or structural rearrangements and/or identification of fetal diseases or conditions. In some embodiments, the methods of prenatal diagnosis described herein can be used to determine de novo balanced chromosomal rearrangements. The methods of prenatal diagnosis described herein can be used alone, or in combination with, the existing prenatal diagnostic methods as discussed earlier.

The term “chromosome” has herein its art understood meaning. It refers to structures composed of very long DNA molecules (and associated proteins) that carry most of the hereditary information of an organism. Chromosomes are divided into functional units called “genes”, each of which contains the genetic code (i.e., instructions) for making a specific protein or RNA molecule. In humans, a normal body cell contains 46 chromosomes; a normal reproductive cell contains 23 chromosomes.

The term “chromosomal abnormality” generally refers to a difference (i.e., a variation) in the number of chromosomes or to a difference (i.e., a modification) in the structural organization or rearrangement of one or more chromosomes as compared to chromosomal number and structural organization in a karyotypically normal individual. As used herein, these terms are also meant to encompass abnormalities taking place at the gene level. The presence of an abnormal number of (i.e., either too many or too few) chromosomes is called “aneuploidy”. Examples of aneuploidy are trisomy 21 and trisomy 13. Structural chromosomal abnormalities include: deletions (e.g., absence of one or more nucleotides normally present in a gene sequence, absence of an entire gene, or missing portion of a chromosome), additions (e.g., presence of one or more nucleotides usually absent in a gene sequence, presence of extra copies of a gene (also called duplication), or presence of an extra portion of a chromosome), rings, inversion, tandem-duplication, translocation breaks and chromosomal rearrangements. Abnormalities that involve deletions or additions of chromosomal material alter the gene balance of an organism and if they disrupt or delete active genes, they generally lead to fetal death or to serious mental and physical defects. Structural rearrangements of chromosomes result from chromosome breakage caused by damage to DNA, errors in recombination, relocation of genomic material between different chromosomes or within the same chromosome, or crossing over the maternal and paternal ends of the separated double helix during meiosis or gamete cell division. In some embodiments, chromosomal rearrangements can be translocations or inversions. A translocation results from a process in which genetic material is transferred from one gene to another. A translocation is balanced when two chromosomes exchange pieces without loss of genetic material, while an unbalanced translocation occurs when chromosomes either gain or lose genetic material. Translocations can involve two chromosomes or only one chromosome. Inversions are produced by a process in which two breaks occur in a chromosome and the broken segment rotates 180°, resulting in the genes being rearranged in reverse order.

In some embodiments, the methods described herein can be used to detect chromosomal micro-abnormalities. As used herein, the term “chromosomal micro-abnormality” refers to a small, subtle and/or cryptic chromosomal abnormality (for example, one involving one or more nucleotides in a gene sequence, or resulting in loss or gain of a single gene copy or one taking place at a subtelomeric region). Examples of chromosomal micro-abnomalities can include, but are not limited to microdeletion, microaddition, microduplication, microrearrangment, microtranslocation, microinversion, subtelomeric rearrangement. As used herein, the terms “microdeletion”, “microaddition”, “microduplication”, “microrearrangement”, “microtranslocation”, “microinversion”, and “subtelomeric rearrangement” refer to chromosomal micro-abnormalities that cannot be detected or are not easily detectable by standard cytogenetic methods, such as, for example, karyotyping analysis.

In some embodiments, the methods described herein can be used to diagnose a disease or condition associated with a chromosomal abnormality or rearrangement. As used herein, the term “disease or condition associated with a chromosomal abnormality or rearrangement” refers to any disease, disorder, condition or defect, which is known or suspected to be caused by a chromosomal abnormality or rearrangement. Exemplary diseases or conditions associated with a chromosomal abnormality include, but are not limited to, trisomies (e.g., Down syndrome, Edward syndrome, Patau syndrome, Turner syndrome, Klinefelter syndrome, and XYY disease), microdeletion/microduplication syndromes, and X-linked disorders (e.g., Duchenne muscular dystrophy, hemophilia A, certain forms of severe combined immunodeficiency, Lesch-Nyhan syndrome, and Fragile X syndrome). Additional examples of diseases or conditions associated with chromosomal abnormalities are given below and may also be found in “Harrison's Principles of Internal Medicine”, Wilson et al. (Ed.), 1991 (12th Ed.), Mc Graw Hill: New York, N.Y., pp 24-46, which is incorporated herein by reference in its entirety.

As used herein, the term “microdeletion/microduplication syndromes” refers to a collection of genetic syndromes that are associated with small or subtle structural chromosomal aberrations, a large number of which are beyond the resolution of detection of standard cytogenetic methods. Microdeletion/microduplication syndromes include, but are not limited to: Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome.

In some embodiments, the methods described herein can be used to diagnose diseases or disorders that have been routinely diagnosed based on analysis of karyotype. As used herein, the term “karyotype” refers to the particular chromosome complement of an individual or a related group of individuals, as defined by the number and morphology of the chromosomes usually in mitotic metaphase. More specifically, a karyotype includes such information as total chromosome number, copy number of individual chromosome types (e.g., the number of copies of chromosome Y) and chromosomal morphology (e.g., length, centromeric index, connectedness and the like). Examination of a karyotype allows detection and identification of chromosomal abnormalities (e.g., extra, missing, or broken chromosomes). However, less extensive or more complex rearrangements of genetic material, chromosomal origins of markers, and subtle translocations are not detectable or are difficult to identify with certainty using karyotyping analyses, which is typically performed with a standard G (or Giemsa) banding staining.

### Methods for Prenatal or Postnatal Determination of Chromosomal Abnormalities

In one aspect, provided herein are methods of diagnosing or determining prenatal chromosomal abnormalities and/or structural rearrangements. The method comprises:

a. subjecting genomic DNA extracted from cells in an amniotic fluid sample to whole-genome sequence analysis using a large-insert jumping library; and
b. identifying, using a specifically-programmed computer system, structural rearrangements or chromosomal breakpoints in the DNA based on the sequencing data, thereby detecting the presence of one or more abnormalities in the genomic DNA of a fetus associated with structural rearrangements or chromosomal breakpoints.

As used herein, the term “whole-genome sequence analysis” refers to analysis of libraries comprising collections of genomic DNA fragments that span large areas (e.g., at least about 30% or more, including, e.g., at least about 40%, at least about 50%, at least about 60%, at least about 70%, at least about 80%, at least about 90%, at least about 95% or more) of the genome. The whole-genome sequence analysis does not imply that the whole genome is necessarily sequenced. However, with sufficient redundancy, the entire genome can be covered, or indeed, covered more than once in a given analysis. In some embodiments, the whole-genome sequence analysis is based on sequencing a library comprising short DNA fragments representing junctions formed form the circularization of larger genomic fragments (termed as a large-insert jumping library). The use of short fragments derived from long genomic inserts allows higher effective genomic coverage while minimizing the cost of whole-genome sequence coverage.

The term “large insert jumping libraries” refers to DNA fragments comprising end sequences derived from genomic DNA, wherein the end sequences are separated by a genomic distance equal to the size of the size-selected DNA. The genomic distance is measured between the endpoints of the size-selected DNA.

As used herein, the term “genomic DNA” refers to sequences of DNA materials or portions thereof isolated from a genome of one or more cells, and include DNAs derived from (i.e., isolated from, amplified from, cloned from as well as synthetic versions of) genomic DNA. Fetal DNA isolated from amniotic fluid may be considered genomic DNA as it was found to represent the entire genome equally.

In some embodiments, the method can further comprise collecting an amniotic fluid sample from a pregnant subject. In these embodiments, the method can further comprising performing amniocentesis. The amniocentesis can be performed on the pregnant subject any time during their pregnancy. In some embodiments, the amniocentesis can be performed on the pregnant subject in the first trimester. In some embodiments, the amniocentesis can be performed on the pregnant subject in the second trimester. In some embodiments, the amniocentesis can be performed on the pregnant subject in the third trimester.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, and avian species, e.g., chicken, emu, ostrich. In certain embodiments of the aspects described herein, the subject is a mammal, e.g., a primate, e.g., a human. In one embodiment, the subject is a mammal. The mammal can be a human, non-human primate, mouse, rat, dog, cat, horse, or cow, but are not limited to these examples. In one embodiment, the subject is a human being. In another embodiment, the subject can be a domesticated animal and/or pet.

In some embodiments, the pregnant subject can have one embryo or fetus in a single pregnancy or have more than one embryo or fetus in a single pregnancy (also known as multiple pregnancy).

In some embodiments, the pregnant woman or the father can have or be suspected of having a chromosomal abnormality or the fetus is suspected of having a disease or condition associated with a chromosomal abnormality. For example, such situations may arise when a previous child of the couple of prospective parents has a chromosomal abnormality, when there is a case of parental chromosomal rearrangement, when there is a case of family history of late-onset disorders with genetic components, when a maternal serum screening test comes back positive, documenting, for example, an increased risk of fetal neural tube defects and/or fetal chromosomal abnormality, or in case of an abnormal fetal ultrasound examination, for example, one that revealed signs known to be associated with aneuploidy.

The methods described herein can generally be applied to a pregnant woman of any age. In some embodiments, the pregnant woman subjected to the methods described herein can be suspected of having a risk of chromosomal abnormality due to advanced maternal age. In these embodiments, the pregnant woman subjected to the methods described herein can be at the age of 35 or above.

Design and Production of a Large-Insert Jumping Library:

In some embodiments, the large-insert jumping library can be created by a process comprising:

a. size-selecting fragments of the genomic DNA;
b. circularizing the size-selected DNA fragments with adaptors comprising a first member of an affinity binding pair and an optional endonuclease recognition site;
c. fragmenting the circularized DNA into linear DNA fragments in the presence of an endonuclease specific for the endonuclease recognition site present in the adaptors or by random shearing the circularized DNA, wherein at least a portion of the linear DNA fragments comprise the adaptors of step (b) and an end sequence derived from the genomic DNA on each end of the linear DNA fragments;
d. contacting the linear DNA fragments with a solid support comprising a second member of the affinity binding pair, thereby selecting linear DNA fragments comprising the first member of the affinity binding pair and the end sequence on either end;
e. amplifying the linear DNA fragments bound to the solid support, thereby generating a library of DNA fragments comprising end sequences derived from the genomic DNA, wherein the end sequences are separated by a genomic distance equal to the size of the size-selected DNA fragments.

Fragments of the genomic DNA in step (a) can be produced by shearing genomic DNA. Fragmenting can also be achieved, for example, by partial endonuclease digestion or partial or complete restriction endonuclease digestion. In some embodiments, the fragments of the genomic DNA can be tightly size-selected at a chosen median. In some applications, it can be advantageous to select different sizes. The DNA fragments can be selected for sizes ranging from about 0.1 kb to about 20 kb, about 0.2 kb to about 15 kb, about 0.5 kb to about 10 kb, about 1 kb to about 10 kb, about 3 kb to about 10 kb, about 5 kb to about 10 kb. In some embodiments, the DNA fragments can be selected for a size of at least about 1 kb or higher, including, e.g., at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, at least about 15 kb or more. In some embodiments, the size-selected DNA fragments can be approximately 2 kb to 6 kb.

In some embodiments, prior to the circularizing the size-selected DNA fragments, method can further comprise end repairing the size-selected DNA fragments. In some embodiments, the size-selected DNA fragments can be end-repaired to acquire blunt ends.

In some embodiments, the method can further comprise ligating to the size-selected DNA fragments adaptors comprising a first member of an affinity binding pair and an optional endonuclease recognition site. In some embodiments, the adaptors can further comprise an oligonucleotide barcode. The oligonucleotide barcode can have a sequence of about 5-1000 nucleotides, about 10-750 nucleotides, about 25-500 nucleotides, about 50-250 nucleotides. In some embodiments, the oligonucleotide barcode can have a sequence of at least about 5 nucleotides, at least about 10 nucleotides, at least about 15 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, at least about 100 nucleotides, at least about 250 nucleotides, at least about 500 nucleotides or more. In some embodiments, the oligonucleotide barcode can be labeled with a detectable label, e.g., but not limited to, a fluorescent label. In some embodiments, the oligonucleotide barcode can be sample-specific, thereby allowing simultaneous amplification of more than one sample in the same amplification reaction. For example, sample-specific oligonucleotide barcodes can vary in nucleotide length and/or sequence. In some embodiments, the sample-specific oligonucleotide barcodes can have distinct detectable labels, e.g., distinct fluorescent labels.

The first member of the affinity binding pair, optional endonuclease, and optional oligonucleotide barcode can be present on the same adaptor or in separate adaptors.

Accordingly, in some embodiments, the circularizing step (b) can be performed in a single reaction or in more than one reaction to ligate adaptors comprising the first member of the affinity binding pair and an optional endonuclease to the size-selected DNA fragments. For example, in some embodiments, the circularizing step (b) can comprise ligating a first adaptor to the size-selected DNA fragments, wherein the first adaptor comprises an endonuclease recognition site; and circularizing the ligated products with a second adaptor comprising a first member of an affinity binding pair. In these embodiments, the first adaptors can be designed to contain overhangs which are complementary to the overhangs of the second adaptors.

While various endonuclease recognition sites known in the art (e.g., sites that are recognized by any commercially-available restriction enzymes, e.g., from New England Biolabs) can be selected for use in the adaptors, in one embodiment, the endonuclease recognition site selected for use in the adaptor comprises an EcoP15I recognition site, and example sequences of first adaptors comprising an EcoP15I recognition site are shown below:

First_adapter_1:
/5Phos/ACAGCAG
First_adapter_2:
/5Phos/CTGCTGTAC

The first member of the affinity binding pair is generally included in the adaptors for subsequent selection of the DNA fragments comprising the circularization junctions. In some embodiments, the first member of the affinity binding pair comprises a biotinylated nucleotide. The term “nucleotide” as used herein generally refers to adenine, guanine, cytosine, thymine, analogs thereof, or derivatives thereof. The term can also encompass nucleic acid-like structures with synthetic backbones.

In some embodiments, the biotinylated nucleotide can comprise a biotinylated thymine. In some embodiments where the biotinylated thymine is added via the second adaptor, example sequences of the second adaptor are shown below:

Second_adaptor_1A:
/5Phos/CGT TC/iBiodT/CCG T
Second_adaptor_2A:
/5Phos/GGA GAA CGG T

The circularized DNA can be circularized DNA can be fragmented to produce linear DNA fragments by endonuclease digestion or random shearing. In some embodiments, the circularized DNA can be fragmented in the presence of an endonuclease enzyme specific for the endonuclease recognition site present, if any, in the adaptors. In some embodiments, the circularized DNA can be fragmented by random shearing (e.g., random acoustic shearing), e.g., to generate larger fragments at the gap junction for longer DNA read lengths at the ends.

At least a portion of the linear DNA fragments comprise the adaptors comprising a first member of an affinity binding pair and an optional endonuclease recognition site, as well as an end sequence on either end of the linear fragments. Each of the end sequences comprises a sequence derived from the genomic DNA. The term “derived from the genomic DNA” refers to sequences of DNA corresponding to nucleotide sequences as present in a genome. In some embodiments, the end sequences can have a nucleotide length of about 10 bp to about 1000 bp of the genomic sequence. In some embodiments, the end sequences can have a nucleotide length of about 15 bp to about 500 bp of the genomic sequence. In some embodiments, the end sequences can have a nucleotide length of about 20 bp to about 250 bp of the genomic sequence. In some embodiments, the end sequences can have a nucleotide length of about 50 bp to about 200 bp of the genomic sequence. In some embodiments, the end sequences can have a nucleotide length of at least about 50 bp, at least about 75 bp, at least about 100 bp, at least about 150 bp, at least about 200 bp, at least about 300 bp, at least about 400 bp, at least about 500 bp, at least about 600 bp, at least about 700 bp, at least about 800 bp, at least about 900 bp, at least about 1000 bp, or more. Without wishing to be bound by theory, there can be no upper limit to the length of the end sequences except that imposed by the sequencing technology.

The linear DNA fragments can then be contacted with a solid support comprising a second member of the affinity binding pair, thereby selecting linear DNA fragments comprising the first member of the affinity binding pair and the end sequence on either end. Where the first member of the affinity binding pair comprises biotin, the second member of the affinity binding pair can comprise streptavidin. The solid support can come in different forms, including, e.g., but not limited to, beads, strips, microplates, sticks, fibers, meshes, or any combinations thereof. The solid support can be made of any materials, e.g., but not limited to, paper, glass, metal, polymer, and any combinations thereof. In some embodiments, the solid support can comprise a magnetic material, e.g., iron oxide. Thus, the solid support can be isolated from an amplification reaction in the presence of a magnetic field after amplification. In some embodiments, the solid support comprising a second member of the affinity binding pair can be magnetic particles coated with the second member of the affinity binding pair. In some embodiments, the solid support can be streptavidin-coated magnetic particles.

The term “affinity binding pair” or “binding pair” refers to first and second molecules that specifically bind to each other. One member of the binding pair is conjugated with first part to be linked while the second member is conjugated with the second part to be linked. As used herein, the term “specific binding” refers to binding of the first member of the binding pair to the second member of the binding pair with greater affinity and specificity than to other molecules.

Exemplary binding pairs include any haptenic or antigenic compound in combination with a corresponding antibody or binding portion or fragment thereof (e.g., digoxigenin and anti-digoxigenin; mouse immunoglobulin and goat antimouse immunoglobulin) and nonimmunological binding pairs (e.g., biotin-avidin, biotin-streptavidin, biotin-neutravidin, hormone [e.g., thyroxine and cortisol-hormone binding protein, receptor-receptor agonist, receptor-receptor antagonist (e.g., acetylcholine receptor-acetylcholine or an analog thereof), IgG-protein A, IgG-protein G, IgG-synthesized protein AG, lectin-carbohydrate, enzyme-enzyme cofactor, enzyme-enzyme inhibitor, and complementary oligonucleotide pairs capable of forming nucleic acid duplexes), and the like. The binding pair can also include a first molecule which is negatively charged and a second molecule which is positively charged.

One example of using binding pair conjugation is the biotin-avidin, biotin-streptavidin or biotin-neutravidin conjugation. In this approach, one of the nucleotide in the adaptor can be biotinylated and the solid support can comprise avidin or streptavidin.

In some embodiments, the method can further comprise adding at least one dA molecule to the end(s) of the linear DNA fragments bound to the solid support.

In some embodiments, the method can further comprise adding a barcode adaptor to the end(s) of the linear DNA fragments bound to the solid support.

The linear DNA fragments bound to the solid support can be amplified by any methods known in the art. In some embodiments, the linear DNA fragments bound to the solid support can be amplified by polymerase chain reaction (PCR).

The large-insert jumping library can then be sequenced. Examples of sequencing technologies are known in the art and can be used herein to sequence the linear DNA fragments, including, but not limited to, paired-end sequencing, de novo sequencing, next-generation sequencing such as massively parallel signature sequencing (MPSS), polony sequencing, pyrosequencing, Illumina (Solexa) sequencing, SOLiD sequencing, ion semiconductor sequencing, DNA nanoball sequencing, Heliscope single molecule sequencing, single molecule real time (SMRT) sequencing), nanopore DNA sequencing, sequencing by hybridization, sequencing with mass spectrometry, microfluidic Sanger sequencing, microscopy-based sequencing techniques, sequencing by synthesis, or any combinations thereof. In some embodiments, the linear DNA fragments bound to the solid support can be amplified by sequencing by synthesis, e.g., but not limited to, Illumina™'s platforms. Exemplary Illumina™'s platforms that can be used for sequencing the large-insert jumping library include, without limitations, HiSeq 2500/1500, HiSeq 2000/1000, Genome Analyzer IIx, MiSeq, HiScanSQ, or any combinations thereof. In some embodiments, Illumina™ HiSeq2000 can be used to sequence the large-insert jumping library.

In some embodiments, the insert sizes (actual genomic distance between read-pair ends in genome coordinate space) are tightly distributed in a unimodal fashion, indicating no contamination from non-junction-spanning read-pairs, as opposed to the Illumina™'s mate-pair method which relies on blunt end ligation at circularization.

In some embodiments, the large-insert jumping library can be performed according to the methods described in Example 2.

Computational Processing:

The sequencing data from the large-insert jumping library is then processed by a specifically-programmed computer system. In some embodiments, the specifically-programmed computer comprises one or more processors; and memory to store one or more programs, the one or more programs comprising instructions for:

a. aligning paired-end sequence reads obtained from the whole-genome sequence analysis against sequence of at least one or more chromosomes;
b. categorizing as anomalous, those read pairs that align to genomic sequences separated by significantly greater than or less than the size of the DNA fragments selected for library creation, that have unexpected orientations, or for which the corresponding end sequences align to different chromosomes;
c. categorizing the anomalous read pairs into the same cluster if both sides of the read pairs align within a selected distance of each other; wherein each output cluster represents a putative structural variant breakpoint; and
d. displaying a content that comprises a signal indicative of information associated with the output clusters, wherein the signal is selected from the group consisting of a signal indicative of one or more detectable structural variant breakpoints; a signal indicative of no detectable structural variant breakpoints; a signal indicative of a normal sample, a signal indicative of a disease or disorder associated with the detectable structural variant breakpoints, and any combination thereof.

In some embodiments, the structural variant breakpoints can be induced by structural rearrangements or chromosomal abnormalities. Examples of structural rearrangements or chromosomal abnormalities includes, but are not limited to, inversion, deletion, translocation, tandem-duplication, excision, insertion, duplicated-insertion, and any combinations thereof.

In some embodiments, the selected distance of step (c) can be determined based on the median and variance of the original size selected fragments as for true structural variants. The sequences from the ends of the fragments will yield paired alignment signals from two different genomic locations that can be ordered and clustered within a length of DNA determined by the size of the fragment and the reference sequence at each genomic location, consistent with a breaking and joining of the genomic locations.

The steps (a)-(d) can be processed and analyzed by one or more functional modules described in Example 3. Each module or sub-modules in Example 3 can be applied alone, or in combinations with other modules described therein or with functionally-equivalent algorithms known in the art, for the whole-genome analysis.

In some embodiments, the specifically-programmed computer can be configured to comprise one or more modules described in Example 3.

In some embodiments, PCR and/or sequencing (e.g., Sanger sequencing) can be used to validate breakpoint clusters generated by the classifier.

In some embodiments, multiple breakpoints representing distinct ends of an event can be linked together using a specific algorithm to discover both simple and complex rearrangements. For example, use of a custom Python script can allow for the discovery of both simple and complex rearrangements.

In some embodiments, variants can be tagged according to sample, and thus ready for inter-sample or inter-study comparisons and/or for further annotation of disease contribution.

In some embodiments, the method can further comprise selecting a treatment plan in response to the disease or condition diagnosed.

Without wishing to be limiting, various embodiments of the methods described herein can be adapted for use in postnatal determination of chromosomal abnormalities. Accordingly, in another aspect, provided herein are methods of diagnosing or determining postnatal chromosomal abnormalities and/or structural rearrangements. The method comprises:

a. subjecting genomic DNA extracted from tissue to whole-genome sequence analysis using a large-insert jumping library; and
b. identifying, using a specifically-programmed computer system, structural rearrangements or chromosomal breakpoints in the DNA based on the sequencing data, thereby detecting the presence of one or more abnormalities in the genomic DNA of a human subject associated with structural rearrangements or chromosomal breakpoints. In some embodiments, the human subject can include a newborn.

Examples of tissue that can be used for extraction of genomic DNA can include, but are not limited to, blood, lung, heart, liver, skin, pancreas, or tissue of any organs. In some embodiments, the tissue for extraction of genomic DNA is a blood sample.

### Systems, e.g., for the Analysis of Whole Genome Sequence Data

Embodiments of a further aspect also provide for systems (and computer readable media for causing computer systems) to, e.g., to analyze whole genome sequence data and/or to preform methods of various aspects described herein. A system comprises:

(a) a determination module configured to receive said at least one test sample and perform at least one sequencing analysis on said at least one test sample;
(b) a storage device configured to store output sequence data from said determination module;
(c) a computing module comprising specifically-programmed instructions to determine from the output sequence data validity or invalidity of structural rearrangements represented by the output sequence data, wherein the instructions comprise:
a. mapping read-pairs of the output sequence data against a reference genome;
b. categorizing the read-pairs into clusters based on at least one common feature;
c. removing the read-pair clusters having their mapping positions localized to predefined centromeric, telomeric, or heterochromatic regions over the reference genome;
d. measuring at least two or more features of the remaining read-pair clusters, wherein said features are selected from the group consisting of:
   (i) number of read-pairs in the cluster;
   (ii) mapping quality scores on both ends of the cluster and the residual between both measurements;
   (iii) read-pair uniqueness across both ends of the cluster and the residual between both measurements;
   (iv) distance between the maximum and minimum mapping positions on both ends of the cluster;
   (v) normalized distance between the maximum and minimum mapping positions on both ends of the cluster;
   (vi) local coverage ratio of the number of the read-pairs in the cluster to the number of proper pairs at a breakpoint junction in the cluster;
   (vii) global coverage ratio of the number of the read-pairs in the cluster to average haploid proper pair coverage in the reference genome;
   (viii) GC percent averaging across regions on both ends of the cluster and the residual between both measurements;
   (ix) alignability percent averaging across sequences of both ends of the cluster and the residual between both measurements; and
   (x) any combinations thereof;
e. performing a pre-trained decision tree classification program to determine the validity or invalidity of the structural rearrangements represented by the clusters; and
   (d) a display module for displaying a content based in part on the data output from said computing module, wherein the content comprises a signal indicative of the presence of valid structural rearrangements, or a signal indicative of the absence of any valid structural rearrangements.

In some embodiments, the sequencing analysis is based on a jumping library. Examples of a jumping library can include, but are not limited to, short jump library, custom barcoded jumping library, long jump library, fosmid-jump library, large-insert jumping library, and any combinations thereof. In some embodiments, the sequencing analysis is based on a large-insert jumping library.

Techniques for nucleic acid sequencing are known in the art and can be used to assay the test sample to determine nucleic acid measurements, for example, but not limited to, paired-end sequencing, de novo sequencing, next-generation sequencing such as massively parallel signature sequencing (MPSS), polony sequencing, pyrosequencing, Illumina (Solexa) sequencing, SOLiD sequencing, ion semiconductor sequencing, DNA nanoball sequencing, Heliscope single molecule sequencing, single molecule real time (SMRT) sequencing), nanopore DNA sequencing, sequencing by hybridization, sequencing with mass spectrometry, microfluidic Sanger sequencing, microscopy-based sequencing techniques, or any combinations thereof. Exemplary Illumina™'s platforms that can be used for sequencing the large-insert jumping library include, without limitations, HiSeq 2500/1500, HiSeq 2000/1000, Genome Analyzer IIx, MiSeq, HiScanSQ, or any combinations thereof. In some embodiments, Illumina™ HiSeq2000 can be used to sequence the large-insert jumping library.

Depending on the nature of test samples and/or applications of the systems as desired by users, the display module can further display additional content. In some embodiments where the test sample is collected or derived from a subject for diagnostic assessment, the content displayed on the display module can further comprise a signal indicative of a diagnosis of a condition (e.g., disease or disorder) or a state of the condition (e.g., disease or disorder) in the subject.

A tangible and non-transitory (e.g., no transitory forms of signal transmission) computer readable medium having computer readable instructions recorded thereon to define software modules for implementing a method on a computer is also provided herein. In one embodiment, the computer readable storage medium comprises: (a) instructions for analyzing the sequencing data stored on a storage device, wherein the analyzing comprises the following: mapping read-pairs of the output sequence data against a reference genome; categorizing the read-pairs into clusters based on at least one common feature; removing the read-pair clusters having their mapping positions localized to predefined centromeric, telomeric, or heterochromatic regions over the reference genome; measuring at least two or more features of the remaining read-pair clusters, wherein said features are selected from the group consisting of: (i) number of read-pairs in the cluster; (ii) mapping quality scores on both ends of the cluster and the residual between both measurements; (iii) read-pair uniqueness across both ends of the cluster and the residual between both measurements; (iv) distance between the maximum and minimum mapping positions on both ends of the cluster; (v) normalized distance between the maximum and minimum mapping positions on both ends of the cluster; (vi) local coverage ratio of the number of the read-pairs in the cluster to the number of proper pairs at a breakpoint junction in the cluster; (vii) global coverage ratio of the number of the read-pairs in the cluster to average haploid proper pair coverage in the reference genome; (viii) GC percent averaging across regions on both ends of the cluster and the residual between both measurements; (ix) alignability percent averaging across sequences of both ends of the cluster and the residual between both measurements; and (x) any combinations thereof; and performing a pre-trained decision tree classification program to determine the validity or invalidity of the structural rearrangements represented by the clusters; and (b) instructions for displaying a content based in part on the data output from the computing module, wherein the content comprises a signal indicative of the presence of valid structural rearrangements, or a signal indicative of the absence of any valid structural rearrangements.

Embodiments of the systems described herein have been described through functional modules, which are defined by computer executable instructions recorded on computer readable media and which cause a computer to perform method steps when executed. The modules have been segregated by function for the sake of clarity. However, it should be understood that the modules need not correspond to discrete blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible media that can be accessed by a computer. Computer readable media includes volatile and nonvolatile, removable and non-removable tangible media implemented in any method or technology for storage of information such as computer readable instructions, data structures, program modules or other data. Computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and nonvolatile memory, and any other tangible medium which can be used to store the desired information and which can accessed by a computer including and any suitable combination of the foregoing.

In some embodiments, the computer readable storage media 700 can include the “cloud” system, in which a user can store data on a remote server, and later access the data or perform further analysis of the data from the remote server.

Computer-readable data embodied on one or more computer-readable media, or computer readable medium 700, may define instructions, for example, as part of one or more programs, that, as a result of being executed by a computer, instruct the computer to perform one or more of the functions described herein (e.g., in relation to system 600, or computer readable medium 700), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system 600, or computer readable medium 700 described herein, may be distributed across one or more of such components, and may be in transition there between.

The computer-readable media can be transportable such that the instructions stored thereon can be loaded onto any computer resource to implement the assays and/or methods described herein. In addition, it should be appreciated that the instructions stored on the computer readable media, or computer-readable medium 200, described above, are not limited to instructions embodied as part of an application program running on a host computer. Rather, the instructions may be embodied as any type of computer code (e.g., software or microcode) that can be employed to program a computer to implement the assays and/or methods described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the system described herein can include a determination module, a storage device, a computing module and a display module. The functional modules can be executed on one, or multiple, computers, or by using one, or multiple, computer networks. The determination module 602 can have computer executable instructions to perform at least one sequencing analysis.

In some embodiments, the determination module 602 can have computer executable instructions to provide sequence information in computer readable form. As used herein, “sequence information” refers to any nucleotide sequence, including but not limited to full-length nucleotide sequences, partial nucleotide sequences, or mutated sequences. Moreover, information “related to” the sequence information includes detection of the presence or absence of a sequence (e.g., detection of a mutation or deletion), determination of the concentration of a sequence in the sample, and the like. The term “sequence information” is intended to include the presence or absence of post-translational modifications (e.g. phosphorylation, glycosylation, summylation, farnesylation, and the like).

As an example, determination modules 602 for determining sequence information may include known systems for automated sequence analysis including but not limited to Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABED 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics FluorImager™ 575, SI Fluorescent Scanners, and Molecular Dynamics FluorImager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England). In some embodiments, the determination module 602 can include an Illumina™ platform, including, without limitations, HiSeq 2500/1500, HiSeq 2000/1000, Genome Analyzer IIx, MiSeq, HiScanSQ, or any combinations thereof.

The sequencing data determined in the determination module can be read by the storage device 604. As used herein the “storage device” 604 is intended to include any suitable computing or processing apparatus or other device configured or adapted for storing data or information. Examples of electronic apparatus suitable for use with the system described herein can include stand-alone computing apparatus, data telecommunications networks, including local area networks (LAN), wide area networks (WAN), Internet, Intranet, and Extranet, and local and distributed computer processing systems. Storage devices 604 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 604 is adapted or configured for having recorded thereon sequence information. Such information may be provided in digital form that can be transmitted and read electronically, e.g., via the Internet, on diskette, via USB (universal serial bus) or via any other suitable mode of communication, e.g., the “cloud”.

As used herein, “stored” refers to a process for encoding information on the storage device 604. Those skilled in the art can readily adopt any of the presently known methods for recording information on known media to generate manufactures comprising the sequence information.

A variety of software programs and formats can be used to store the sequence information on the storage device. Any number of data processor structuring formats (e.g., text file or database) can be employed to obtain or create a medium having recorded thereon the sequence information.

By providing sequence information in computer-readable form, one can use the sequence information in readable form (e.g., as a multi-dimensional vector) in the computing module 606 to perform the whole-genome analysis as described herein. The analysis made in computer-readable form provides a computer readable analysis result which can be processed by a variety of means. Content 608 based on the analysis result can be retrieved from the computing module 606 to indicate the presence or absence of structural rearrangements or chromosomal abnormalities.

In one embodiment, the storage device 604 to be read by the computing module 606 can comprise sequences of reference genome, e.g., sequences of a human genome.

The “computing module” 606 can use a variety of available software programs and formats to analyze whole genome sequence data. The computing module 606 can be configured to perform one or more modules as described in Example 3.

The computing module 606, or any other module of the system described herein, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular embodiment, users can directly access data (via Hypertext links for example) residing on Internet databases using a HTML interface provided by Web browsers and Web servers. In another embodiment, users can directly access data residing on the “cloud” provided by the cloud computing service providers.

The computing module 606 provides computer readable analysis result that can be processed in computer readable form by predefined criteria, or criteria defined by a user, to provide a content based in part on the analysis result that may be stored and output as requested by a user using a display module 610. The display module 610 enables display of a content 608 based in part on the comparison result for the user, wherein the content 608 is a signal indicative of the presence of valid structural rearrangements, a signal indicative of the absence of any valid structural arrangements, a signal indicative of a disease or disorder associated with the structural arrangements, and any combinations thereof. Such signal, can be for example, a display of content 608 on a computer monitor, a printed page of content 608 from a printer, or a light or sound.

In various embodiments of the computer system described herein, the computing module 606 can be integrated into the determination module 602.

In one embodiment, the content 608 based on the analysis result is displayed a on a computer monitor. In one embodiment, the content 608 based on the analysis result is displayed through printable media. The display module 610 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content 608 based on the analysis result. It should be understood that other modules of the system described herein can be adapted to have a web browser interface. Through the Web browser, a user may construct requests for retrieving data from the computing module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to the physiological state of a target cell in a test sample, e.g., display of an indication of the presence or absence of the selected reference phenotype in a target cell, or display of information based thereon. In one embodiment, the information of the reference sample data is also displayed.

In any embodiments, the computing module can be executed by a computer implemented software as discussed earlier. In such embodiments, a result from the computing module can be displayed on an electronic display. The result can be displayed by graphs, numbers, characters or words. In additional embodiments, the results from the computing module can be transmitted from one location to at least one other location. For example, the comparison results can be transmitted via any electronic media, e.g., internet, fax, phone, a “cloud” system, and any combinations thereof. Using the “cloud” system, users can store and access personal files and data or perform further analysis on a remote server rather than physically carrying around a storage medium such as a DVD or thumb drive.

The system 600, and computer readable medium 700, are merely illustrative embodiments, e.g., for identifying a physiological state of a target cell and/or for use in the methods of various aspects described herein and is not intended to limit the scope of the inventions described herein. Variations of system 600, and computer readable medium 700, are possible and are intended to fall within the scope of the inventions described herein.

The modules of the machine, or used in the computer readable medium, may assume numerous configurations. For example, function may be provided on a single machine or distributed over multiple machines.

Other Applications of the Methods and/or Systems Described Herein

In some embodiments, the methods, and systems described herein can provide a service to physicians that will enable the physicians to tailor their recommendation for prenatal care or treatment. Stated another way, in some embodiments, the methods, and systems described herein can be performed by one or more service providers, e.g., a diagnostic laboratory to assay a biological sample taken from a subject and perform the assay analysis, or a diagnostic laboratory to assay a biological sample taken from a subject and then provide the assay results to a third-party for the assay analysis. For example, a biological sample (e.g., an amniotic fluid sample) taken from a subject, e.g., by a skilled practitioner, can be sent to a laboratory facility (e.g., a CLIA-certified laboratory), for example, one such lab is operated by Quest Diagnostics. The laboratory may assay the biological sample to perform the whole genome sequence analysis and then analyze the assay results with respect to a reference in accordance with one or more embodiments of the methods described herein. In some embodiments, the laboratory can assay the biological sample and then send the assay results to a third-party for the analysis, e.g., providing a report to the practitioner, who can make an appropriate decision on a treatment regimen.

Chromosomal Abnormalities and Associated Diseases and Conditions Amenable to Prenatal or Postnatal Diagnosis Using Methods and/or Systems or Various Aspects Described Herein

Different embodiments of the methods and/or systems described herein can be used for prenatal diagnosis of a disease or disorder associated with chromosomal abnormalities or structural rearrangement. Structural chromosomal abnormalities include, but are not limited to, deletions (e.g., absence of one or more nucleotides normally present in a gene sequence, absence of an entire gene, or missing portion of a chromosome), additions (e.g., presence of one or more nucleotides normally absent in a gene sequence, presence of extra copies of genes (also called duplications), or presence of an extra portion of a chromosome), rings, breaks, and chromosomal rearrangements, such as translocations and inversions.

In some embodiments, the methods and/or systems described herein can be used to detect chromosomal abnormalities involving the X chromosome. A large number of these chromosomal abnormalities are known to be associated with a group of diseases and conditions collectively termed X-linked disorders. For example, the methods and/or systems described herein can be used to detect mutations in the HEMA gene on the X chromosome (Xq28), which are associated with Hemophilia A, a hereditary blood disorder, primarily affecting males and characterized by a deficiency of the blood clotting protein known as Factor VIII resulting in abnormal bleeding.

In some embodiments, the methods and/or systems described herein can be used to detect mutations in the DMD gene on chromosome X (Xp21.2), that cause dystrophinopathies such as Duchenne muscular dystrophy. Duchenne muscular dystrophy, which occurs with an incidence rate of approximately 1 in 3,000 live-born male infants, is characterized by progressive muscle weakness starting as early as 2 years of age.

In some embodiments, the methods and/or systems described herein can be used to detect mutations in the HPRT1 gene located at position q26-q27.2 on the X chromosome. This chromosomal abnormality is associated with Lesch-Nyhan syndrome, a rare disease which involves disruption of the metabolism of purines. Lesch-Nyhan syndrome is characterized by neurologic dysfunction, cognitive and behavioral disturbances, and uric acid overproduction.

In some embodiments, the methods and/or systems described herein can be used to detect mutations in the IL2RG gene at chromosomal location Xq13.1, that are responsible for half of all severe combined immunodeficiency cases. Severe combined immunodeficiency represents a group of rare, sometimes fatal, congenital disorders characterized by little or no immune response. Certain forms of severe combined immunodeficiency are also associated with a mutation in JAK3 (an important signaling molecule activated by IL2RG), located on chromosome 19; other forms result from chromosomal abnormalities involving the ADA gene on chromosome 20.

In some embodiments, the methods and/or systems described herein can be used to detect an amplification (presence of more than 200 copies) of a CGG motif at one end of the FMR1 gene (Xq27.3) on the X chromosome, which is associated with Fragile X syndrome, the most common inherited form of mental retardation currently known and whose effects are seen more frequently and with greater severity in males than in females.

In some embodiments, the methods and/or systems described herein can be used to detect other diseases or conditions known to be associated with amplifications of nucleotide motifs. For example, myotonic dystrophy, which is a multisystem disorder that affects skeletal muscle and smooth muscle, as well as the eye, heart, endocrine system, and central nervous system, is associated with over-amplification of a CTG motif (>37 copies) on the DMPK gene on chromosome 19 (19q13.2-q13.3). Another example is spinobulbar muscular atrophy, which is a gradually progressive neuromuscular disorder that affects only males, and is associated with amplification of a CAG repeat (>35 copies) in the androgen receptor (AR) gene located on chromosome 11 (Xq11-q12).

In addition to Fragile X syndrome, a number of other retardation disorders are known to result from chromosomal abnormalities involving the terminal regions (or tips) of chromosomes (i.e., telomeres). A large part of the DNA sequence of telomeres are shared among different chromosomes. However telomeres also comprise a unique (much smaller) sequence region that is specific to each chromosome and is very gene-rich (S. Saccone et al, Proc. Natl. Acad. Sci. USA, 1992, 89: 4913-4917). Chromosome rearrangements involving telomeric regions can have serious clinical consequences. For example, submicroscopic subtelomeric chromosome rearrangements have been found to be a significant cause of mental retardation with or without congenital anomalies (J. Flint et al, Nat. Genet. 1995, 9: 132-140; S. J. L. Knight et al, Lancet, 1999, 354: 1676-1681; B. B. de Vries et al, J. Med. Genet. 2001, 38: 145-150; S. J. L. Knight and J. Flint, J. Med. Genet. 2000, 37: 401-409). Telemore regions have the highest recombination rate and are prone to aberrations resulting from illegitimate pairing and crossover. Since the terminal portions of most chromosomes appear nearly identical by routine karyotyping analysis at the 450- to 500-band level, detection of chromosomal rearrangements in these regions is difficult using standard methodologies. In some embodiments, the methods and/or systems described herein can be used to detect chromosome arrangements involving telomeric regions and/or diagnose diseases or conditions associated with the same.

Diseases and conditions associated with telomeric abnormalities include, for example, Cri du Chat syndrome, a disease that may account for up to 1% of individuals with severe mental retardation and which is characterized by deletion of the distal portion of chromosome 5. Another example is Wolf-Hirschhorn syndrome, a disorder that is characterized by typical facial features and microcephaly, and may also be accompanied by skeletal anomalies, congenital heart defects, hearing loss, urinary tract malformations and structural brain abnormalities. Wolf-Hirschhorn syndrome is associated with deletion of the distal portion of the short arm of chromosome 4 involving band 4p16. In certain cases, this deletion occurs along with other chromosomal abnormalities such as a ring or unbalanced translocation involving chromosome 4.

In some embodiments, the methods and/or systems described herein can also find applications in basic and clinical research investigations aimed at acquiring a better understanding of the role of subtelomeric rearrangements in a number of conditions associated with mental retardation.

In some embodiments, the methods and/or systems described herein may also be used to detect chromosomal abnormalities associated with microdeletion/microduplication syndromes. Microdeletion/microduplication syndromes are a collection of genetic syndromes that are associated with small, cryptic or subtle chromosomal structural aberrations (S. K. Shapira, Curr. Opin. Pediatr. 1998, 10: 622-627), a large number of which are beyond the resolution of detection of standard cytogenetic methods. Some microdeletion syndromes are caused by loss of a single gene; others involve multiple genes or an unknown number of genes. Others still are considered contiguous gene deletion syndromes where deletion of physically contiguous genes leads to complex phenotypic abnormalities.

In some embodiments, the methods and/or systems described herein can be used to detect deletion of segment q11-q13 on chromosome 15, which, when it takes place on the paternally derived chromosome 15, is associated with Prader-Willi syndrome (a disorder characterized by mental retardation, decreased muscle tone, short stature and obesity) and which, when it happens on the maternally derived chromosome 15, is linked to Angelman syndrome (a neurogenetic disorder characterized by mental retardation, speech impairment, abnormal gait, seizures and inappropriate happy demeanor).

In some embodiments, the methods and/or systems described herein can be used to detect microdeletions in chromosome 22, for example those occurring in band 22q11.2, which are linked to DiGeorge syndrome, an autosomal dominant condition that is found in association with approximately 10% of cases in prenatally-ascertained congenital heart disease.

In some embodiments, the methods and/or systems described herein can be used to diagnose Smith-Magenis syndrome, the most frequently observed microdeletion syndrome. Smith-Magenis syndrome is characterized by mental retardation, neuro-behavorial anomalies, sleep disturbances, short stature, minor craniofacial and skeletal anomalies, congenital heart defects and renal anomalies. It is associated with an interstitial deletion of the chromosome band 17p11.2.

In some embodiments, the methods and/or systems described herein can also be used to detect a microdeletion involving the CREBBP gene on chromosome 16 (16p13.3), which is associated with Rubinstein-Taybi syndrome, a disorder characterized by moderate-to-severe mental retardation, distinctive facial features and short stature.

In some embodiments, the methods and/or systems described herein can also be used to detect micro-rearrangements within the LIS1 gene in chromosome band 17p13.3, which are associated with Miller-Dieker syndrome, a multiple malformation disorder characterized by classical lissencephaly (i.e., smooth brain), a characteristic facial appearance and sometimes other birth defects. Miller-Dieker syndrome is considered a contiguous gene deletion syndrome. In Miller-Dieker patients, a deletion of the LIS1 gene is always accompanied with telomeric loci in excess of 250 kb.

In some embodiments, the methods and/or systems described herein can also be used to detect a deletion at location q11.23 on chromosome 7, which is associated with Williams syndrome, a developmental disorder that includes cardiovascular abnormalities, dysmorphic facial features, developmental delay with a unique cognitive profile, infantile hypercalcaemia and growth retardation.

In some embodiments, the methods and/or systems described herein can be used to diagnose a disease or condition associated with multiple different chromosomal abnormalities. For example, Charcot-Marie-Tooth (CMT) hereditary neuropathy refers to a group of disorders characterized by a chronic motor and sensory polyneuropathy and associated with chromosomal abnormalities involving the PMPP2 gene on chromosome 17 (17p11.2), the MPZ gene on chromosome 1 (1q22), the NEFL gene on chromosome 8 (8q21), the GJB1 gene on chromosome X (Xq13.1), the EGR2 gene on chromosome 10 (10q21.1-q22.1), and the PRX gene on chromosome 19 (19q13.1-q13.2).

Other chromosomal abnormalities that can be detected and identified by the methods and/or systems described herein include, for example, a segmental duplication of a subregion on chromosome 21 (such as 21q22), which can be present on chromosome 21 or another chromosome (i.e., after translocation) and is associated with Down syndrome.

In some embodiments, the methods and/or systems described herein can also be used to detect a deletion of a gene called Rb on chromosome 13 (13q14), which is associated with the hereditary form of retinoblastoma. Retinoblastoma occurs in early childhood and leads to the formation of tumors in both eyes. Left untreated, retinoblastoma is most often fatal. However, a survival rate over 90% is achieved with early post-natal diagnosis and modern methods of treatment.

In some embodiments, the methods and/or systems described herein can also be used to detect a point mutation in the HBB gene found on chromosome 11 (lip 15), which is associated with sickle cell anemia, the most common inherited blood disease in the US. Symptoms of sickle cell anemia include chronic hemolytic anemia and severe infections, as well as episodes of pain.

In some embodiments, the methods and/or systems described herein can be used to detect deletions involving chromosomal region lip 13, which are known to be associated with different syndromes such as Wilms tumor (a cancer of the kidneys affecting children), aniridia (a disease of the eyes), genitourinary malformation, and mental retardation.

In some embodiments, the methods and/or systems described herein can be used to detect chromosomal abnormalities affecting the GAB gene on chromosome 1 (1q21), which are known to be associated with Gaucher disease, an inherited illness which encompasses a continuum of clinical findings from a prenatal-lethal form to an asymptomatic form.

Without wishing to be limiting, the methods and/or systems described herein can also be used to detect abnormal number of chromosomes, such as those in which there is an extra set(s) of the normal (or haploid) number of chromosomes (triploidy and tetraploidy), those with a missing individual chromosome (monosomy) and those with an extra individual chromosome (trisomy and double trisomy). The presence of an abnormal number of chromosomes in an otherwise diploid organism is called aneuploidy (see, A. C. Chandley, in: “Human Genetics—Part B: Medical Aspects”, 1982, Alan R. Liss: New York, N.Y.). Approximately half of spontaneous abortions are associated with the presence of an abnormal number of chromosomes in the karyotype of the fetus (M. A. Abruzzo and T J. Hassold, Environ. Mol. Mutagen. 1995, 25: 38-47), which makes aneuploidy the leading cause of miscarriage. Trisomy is the most frequent type of aneuploidy and occurs in 4% of all clinically recognized pregnancies (T J. Hassold and PA. Jacobs, Ann. Rev. Genet. 1984, 18: 69-97). The most common trisomies involve the chromosomes 21 (associated with Down syndrome), 18 (Edward syndrome) and 13 (Patau syndrome) (see, for example, G. E. Moore et al, Eur. J. Hum. Genet. 2000, 8: 223-228). Other aneuploidies are associated with Turner syndrome (presence of a single X chromosome), Klinefelter syndrome (characterized by an XXY karyotype) and XYY disease (characterized by an XYY karyotype).

Accordingly, in some embodiments, the methods and/or systems described herein can be used to diagnose diseases and conditions associated with aneuploidies including, but not limited to: Down syndrome, Edward syndrome and Patau syndrome, as well as Turner syndrome, Klinefelter syndrome and XYY disease.

### SOME SELECTED DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with numerical values means±1%.

In one aspect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The term “normal healthy subject” refers to a subject who has no symptoms of any diseases or disorders, or who is not identified with any diseases or disorders, or who is not on any medication treatment, or a subject who is identified as healthy by physicians based on medical examinations.

As used herein, the term “administer” refers to the placement of a composition into a subject by a method or route which results in at least partial localization of the composition at a desired site such that desired effect is produced. Routes of administration suitable for the methods described herein can include both local and systemic administration. Generally, local administration results in a higher amount of a therapeutic agent being delivered to a specific location (e.g., a target site to be treated) as compared to the entire body of the subject, whereas, systemic administration results in delivery of a therapeutic agent to essentially the entire body of the subject.

Embodiments of various aspects described herein can be defined in any of the following numbered paragraphs:
1. A method of prenatal determination of chromosomal abnormalities comprising:
   a. subjecting genomic DNA extracted from cells in an amniotic fluid sample to whole-genome sequence analysis using a large-insert jumping library; and
   b. identifying, using a specifically-programmed computer system, structural rearrangements or chromosomal breakpoints in the DNA based on the sequencing data, thereby detecting the presence of one or more abnormalities in the genomic DNA of a fetus associated with structural rearrangements or chromosomal breakpoints.
2. A method of postnatal determination of chromosomal abnormalities comprising:
   a. subjecting genomic DNA extracted from tissue to whole-genome sequence analysis using a large-insert jumping library; and
   b. identifying, using a specifically-programmed computer system, structural rearrangements or chromosomal breakpoints in the DNA based on the sequencing data, thereby detecting the presence of one or more abnormalities in the genomic DNA of a human subject associated with structural rearrangements or chromosomal breakpoints.
3. The method of paragraph 1 or 2, wherein the large-insert jumping library is created by a process comprising:
   a. size-selecting fragments of the genomic DNA;
   b. circularizing the size-selected DNA fragments with adaptors comprising a first member of an affinity binding pair, and an optional endonuclease recognition site;
   c. fragmenting the circularized DNA into linear DNA fragments in the presence of an endonuclease specific for the endonuclease recognition site present in the adaptors or by random shearing the circularized DNA, wherein at least a portion of the linear DNA fragments comprise the adaptors of step (b) and an end sequence derived from the genomic DNA on each end of the linear DNA fragments;
   d. contacting the linear DNA fragments with a solid support comprising a second member of the affinity binding pair, thereby selecting linear DNA fragments comprising the first member of the affinity binding pair and the end sequence on either end;
   e. amplifying the linear DNA fragments bound to the solid support, thereby generating a library of DNA fragments comprising end sequences derived from the genomic DNA, wherein the end sequences are separated by a genomic distance equal to the size of the size-selected DNA fragments.
4. The method of paragraph 3, wherein the size-selected DNA fragments are approximately 2 kb to 6 kb.
5. The method of paragraph 3 or 4, wherein the optional endonuclease recognition site is EcoP15I restriction site.
6. The method of any of paragraphs 3-5, wherein the first member of the affinity binding pair comprises a biotinylated nucleotide.
7. The method of paragraph 6, wherein the second member of the affinity binding pair comprises streptavidin.
8. The method of any of paragraphs 3-7, wherein the end sequence on either end has a length of about 50 bp to about 200 bp of the genomic sequence.
9. The method of any of paragraphs 3-8, wherein the amplifying of step (e) is performed by polymerase chain reaction.
10. The method of any of paragraphs 3-9, wherein the oligonucleotide barcode is sample-specific, thereby allowing simultaneous amplification of more than one sample in the same amplification reaction.
11. The method of any of paragraphs 1-10, wherein the specifically-programmed computer system comprises one or more processors; and memory to store one or more programs, the one or more programs comprising instructions for:
   a. aligning paired-end sequence reads obtained from the whole-genome sequence analysis against sequence of at least one or more chromosomes;
   b. categorizing as anomalous, those read pairs that align to genomic sequences separated by significantly greater than or less than the size of the DNA fragments selected for library creation, that have unexpected orientations, or for which the corresponding end sequences align to different chromosomes;
   c. categorizing the anomalous read pairs into the same cluster if both sides of the read pairs align within a selected distance of each other; wherein each output cluster represents a putative structural variant breakpoint; and
   d. displaying a content that comprises a signal indicative of information associated with the output clusters, wherein the signal is selected from the group consisting of a signal indicative of one or more detectable structural variant breakpoints; a signal indicative of no detectable structural variant breakpoints; a signal indicative of a normal sample, a signal indicative of a disease or disorder associated with the detectable structural variant breakpoints, and any combination thereof.
12. The method of paragraph 11, wherein the structural variant breakpoints are induced by structural rearrangements selected from the group consisting of inversion, deletion, translocation, excision, insertion, duplicated-insertion, tandem-duplication, and any combinations thereof
13. The method of any of paragraphs 1-12, wherein the sequencing is performed on an Illumina™ platform.
14. A system comprising
   (a) a determination module configured to receive said at least one test sample and perform at least one sequencing analysis on said at least one test sample;
   (b) a storage device configured to store output sequence data from said determination module;
   (c) a computing module comprising specifically-programmed instructions to determine from the output sequence data validity or invalidity of structural rearrangements represented by the output sequence data, wherein the instructions comprise:
   mapping read-pairs of the output sequence data against a reference genome;
   categorizing the read-pairs into clusters based on at least one common feature;
   removing the read-pair clusters having their mapping positions localized to predefined centromeric, telomeric, or heterochromatic regions over the reference genome;
   measuring at least two or more features of the remaining read-pair clusters, wherein said features are selected from the group consisting of:
      (i) number of read-pairs in the cluster;
      (ii) mapping quality scores on both ends of the cluster and the residual between both measurements;
      (iii) read-pair uniqueness across both ends of the cluster and the residual between both measurements;
      (iv) distance between the maximum and minimum mapping positions on both ends of the cluster;
      (v) normalized distance between the maximum and minimum mapping positions on both ends of the cluster;
      (vi) local coverage ratio of the number of the read-pairs in the cluster to the number of proper pairs at a breakpoint junction in the cluster;
      (vii) global coverage ratio of the number of the read-pairs in the cluster to average haploid proper pair coverage in the reference genome;
      (viii) GC percent averaging across regions on both ends of the cluster and the residual between both measurements;
      (ix) alignability percent averaging across sequences of both ends of the cluster and the residual between both measurements; and
      (x) any combinations thereof;
      performing a pre-trained decision tree classification program to determine the validity or invalidity of the structural rearrangements represented by the clusters; and
   (d) a display module for displaying a content based in part on the data output from said computing module, wherein the content comprises a signal indicative of the presence of valid structural rearrangements, or a signal indicative of the absence of any valid structural rearrangements.
15. The system of paragraph 14, wherein the sequencing analysis is based on a large-insert jumping library.

### Examples

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

### Clinical Diagnosis by Whole-Genome Sequencing of a Prenatal Sample

### Case Report

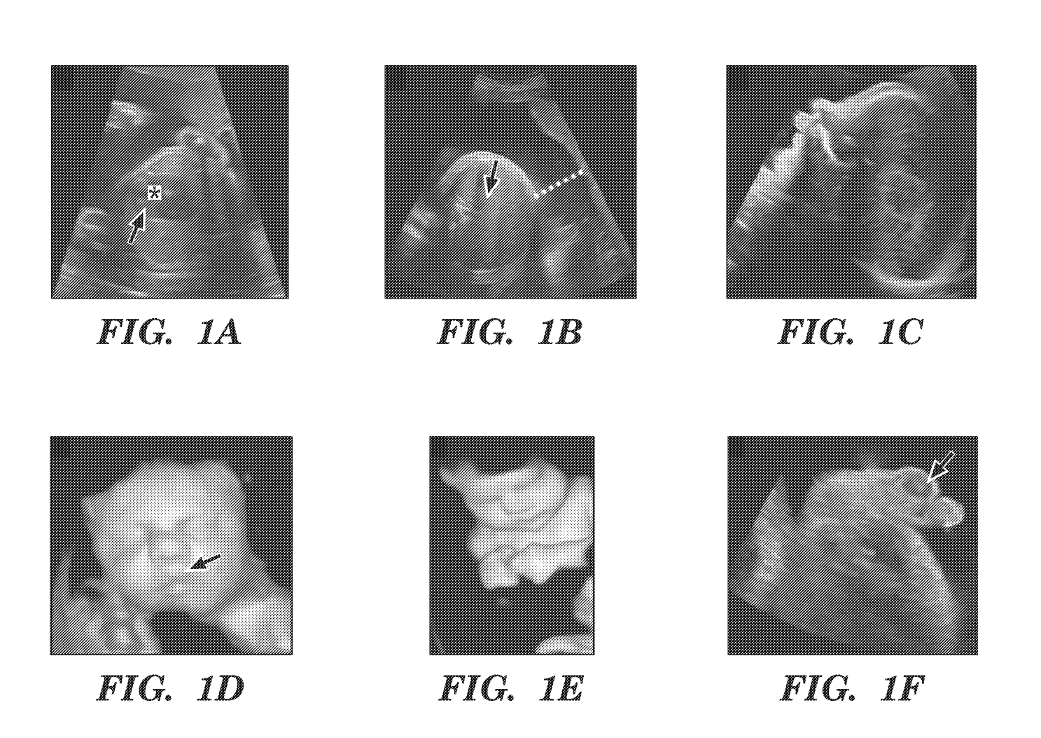

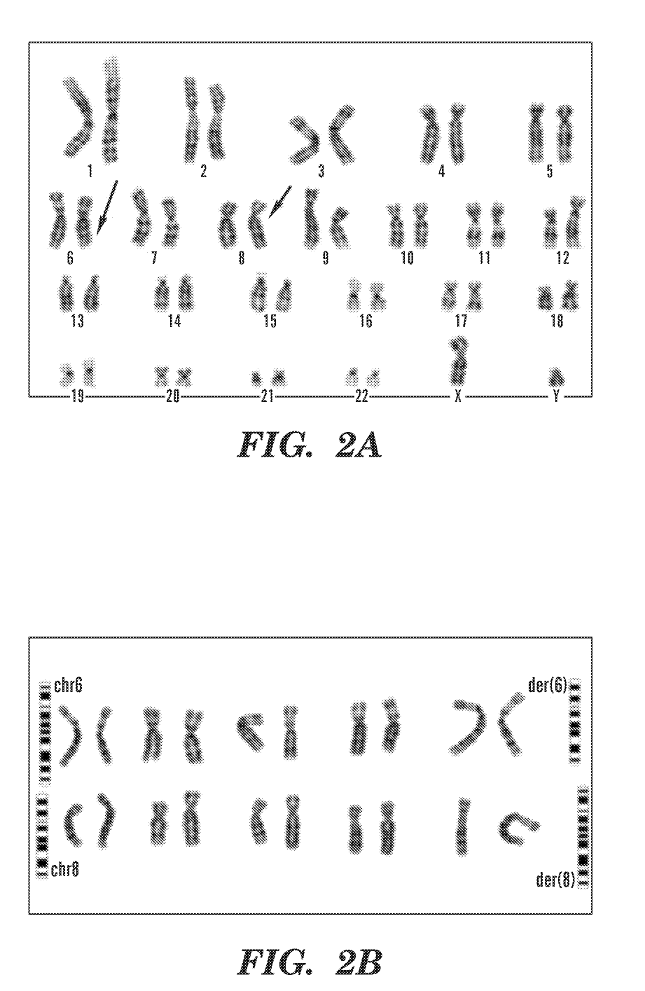

A pregnant 37-year-old woman with a history of infertility and spontaneous abortion and no previous full-term pregnancy presented after ultrasonography performed at 18.8 weeks of gestation revealed fetal abnormalities, including a hypoplastic right ventricle and tricuspid atresia (FIG. 1A). Conception had occurred after the fourth cycle of in vitro fertilization, and the results of ultrasonography and genetic screening performed during the first trimester had been normal. A pediatric cardiology review included the consideration of two or three surgeries for possible palliation, as well as pregnancy termination. Follow-up fetal surveys revealed a level of amniotic fluid that was elevated but within the normal range at 27.3 weeks and polyhydramnios at 30.4 weeks; a small, intermittently undetected stomach was also noted (FIG. 1B). Esophageal atresia was considered along with the possibility of surgical repair after delivery. At 33.3 weeks' gestation, additional findings on fetal ultrasonography indicated the possibility of micrognathia, flexed extremities, and severe polyhydramnios, indicating a differential diagnosis that included arthrogryposis, the Stickler syndrome, and trisomy 18 (FIGS. 1C, 1D, and 1E). Therapeutic amnioreduction was performed, and 20 ml of fluid was submitted for cytogenetic analysis, with a portion of the fluid saved for possible array-based comparative genomic hybridization (CGH) testing. Karyotyping with Giemsa (GTG) banding revealed an apparently balanced de novo translocation, 46,XY,t(6; 8)(q13; q13)dn (FIGS. 2A-2B). Abnormal findings detected on fetal magnetic resonance imaging at 34.4 weeks' gestation included moderately severe polyhydramnios, the absence of a fluid-filled stomach, a nondilated esophagus to the level of the carina, microstomia, an enlarged protruding superior lip, intermittent abnormal swallowing motion with mild protrusion of the tongue, and abnormal fetal position, with flexed elbows and knees, abducted hips, and clenched hands.

A medical genetics consultation at 34.4 weeks included discussion of the possibility of a syndrome resulting from the disruption of one or more genes, microdeletions or microduplications in the breakpoint regions created during an apparently balanced chromosome rearrangement, or a combination of these abnormalities. An array-based CGH analysis revealed no clinically significant loss or gain of genetic material (Table 1).

TABLE 1
Copy number variants identified by array CGH

| Bands | Genomic Coordinates | Minimum Size | Type* |
|---|---|---|---|
| 6p21.1 | Chr 6: 43137563-43181981 | 44.42 kb | Gain |
| 8p11.23 | Chr 8: 39365946-39490567 | 124.62 kb | Loss |
| 11q25 | Chr 11: 132338283-132769193 | 430.91 kb | Gain |
| 17q21.31 | Chr 17: 41541432-41631306 | 89.87 kb | Gain |
| Xp22.33/ | Chr X: 856620-1164762 | 308.14 kb | Gain |
| Yp11.32 | | | |

*No results reported as clinically significant

The results of ultrasonography performed at 35.3 weeks indicated the possibility of an undescended right testicle. Absence of fetal movement during ultrasonography at 36.2 weeks led to an immediate cesarean section. At the time of the cesarean section, polyhydramnios was observed and the baby was found to have neurologic and respiratory depression, prompting intubation after delivery. On the basis of clinical features, the infant received a postnatal diagnosis of the CHARGE syndrome (Online Mendelian Inheritance in Man [OMIM] number, 214800). Plans for any immediate surgeries were postponed until after stabilization, but the infant's clinical condition worsened, and he died at 10 days of age.

### Exemplary Methods

Sequencing Analysis.

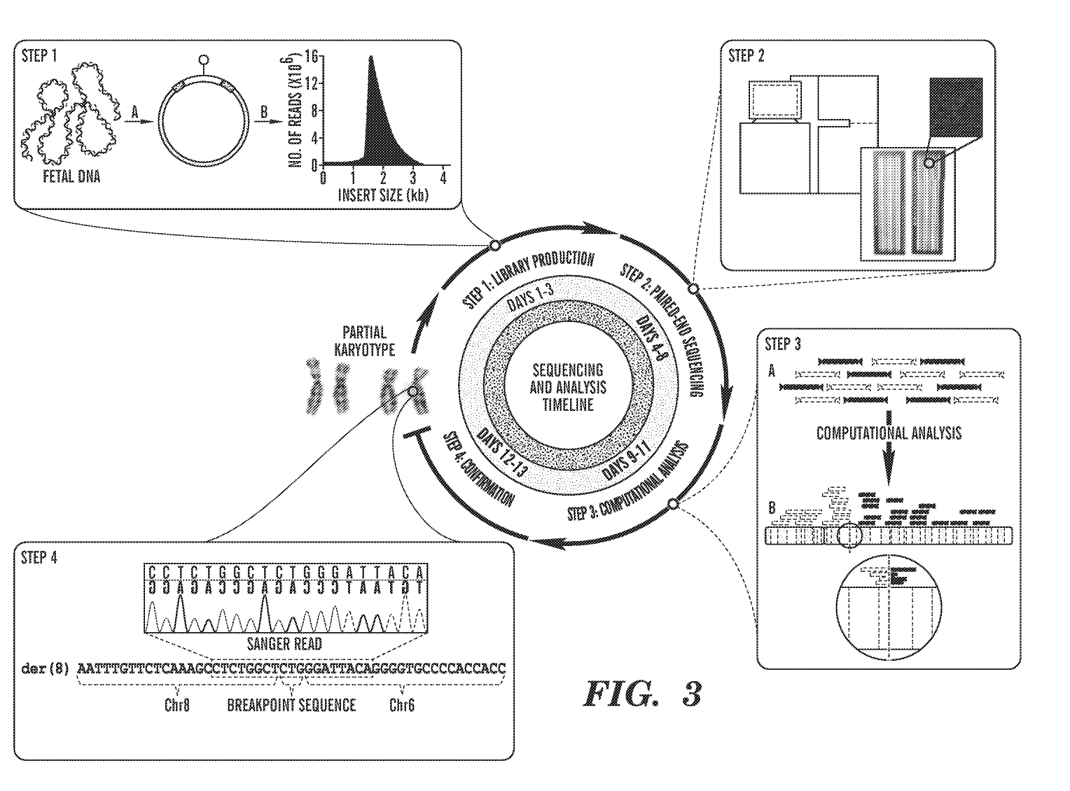

The paired ends of approximately 220-bp DNA fragments separated by approximately 2 kb of contiguous genomic DNA were sequenced [6, 7]. The entire four-step, 13-day process is shown in FIG. 3.

On days 1 through 3, genomic DNA was sheared and selected according to size such that the majority of DNA fragments were approximately 2 kb. These fragments were circularized with adapters containing an EcoP15I recognition site and a biotinylated thymine. The circularized DNA was processed into fragments by means of a restriction digest, and the fragments at the circularization junction were retained by binding the biotinylated thymine to streptavidin beads. Genomic libraries suitable for next-generation sequencing on an Illumina platform were created from these fragments (which spanned the circularization junction) while they were bound to the streptavidin beads, yielding a library of DNA fragments with ends separated by a genomic distance equal to the size of the circularized fragments (FIG. 3) [7]. On days 4 through 8, paired-end, 25-cycle sequencing was performed on a single lane of an Illumina HiSeq 2000.

During computational and statistical analysis, on days 9 through 11, reads were aligned with the use of the Burrows-Wheeler alignment tool [8], and BAM files were then processed with a C++ program (BamStat) to tabulate mapping statistics and output lists of anomalous read pairs (i.e., ends that map to two different chromosomes, abnormally sized inserts, or unexpected strand orientations)[7]. Mapping and assembly artifacts were excluded [9-11] to elucidate chimeric read pairs. These chimeric read pairs provided possible candidate translocation “clusters” throughout the genome. Read-pair clustering and translocation discovery were performed by two independent analysts with knowledge only of the chromosomes involved in the translocation.

Analyses were performed by first inverting all read pairs from standard outward facing orientation that result from sequencing toward the circularization junction for large insert or “jumping” libraries. Candidate clusters were defined as a grouping of chimeric read pairs in which positions of the anomalous mapping fragment ends aligned within 3,760 bp (five standard deviations outside of the median fragment size) of more than eight other chimeric read pairs with high mapping quality (Q20 or greater) and contained an average of at least 90% unique reads from each end. Otherwise stated, fragments with sequenced ends that mapped to two different chromosomes were required to cluster near each other, a candidate cluster contained more than 25% of the expected read depth for a true event based on 34-fold coverage per chromosome, and the same read(s) could not be duplicated many times

On days 12 and 13, DNA was amplified from cells obtained from the amniotic fluid with the use of polymerase-chain-reaction (PCR) primers designed according to the sequence reads supporting the translocation junction. The amplified products were then sequenced. Table 2 shows the translocation amplification primers designed based on the translocation junction sequences (Table 3).

TABLE 2
Examples of translocation amplification
primers based on the translocation
junction sequences shown in Table 3.

| Deriv- | PCR | | | Start | PCR | | | Start |
|---|---|---|---|---|---|---|---|---|
| ative | Primer F | Chr | Strand | (bp) | Primer R | Chr | Strand | (bp) |
| der(6) | GTCCTGAGCTCATC | 6 | + | 70404849 | ATGCCAAAAAGTGT | 8 | − | 61629445 |
| | CTTTATAGCCAG | | | | GCTACAGAG | | | |
| der(8) | GTTGCTGACATAGC | 8 | + | 61627425 | GCGTGAATCATTCA | 6 | − | 70406713 |
| | TCTATCAGAAAGG | | | | TGAATACCATTATG | | | |

F = forward,
R = reverse

TABLE 3
Sequences of the translocation junctions

| Derivative | Junction Fragment Sequence |
|---|---|
| der(6) | GTCCTGAGCTCATCCTTTATAGCCAGTTGAGATA |
| | ATTATACAGATATGCCACTTCTTGCCTACCCTCA |
| | GATGTAACTTGTTACTATCTGAGGCCATATCCTT |
| | GCTTCCTTCTTTGCAATGTACAGAAGGAAATATC |
| | CCTCATTCTATCTAAAGCATTCTGTCCTGGATAT |
| | TAATACCATATTGCGCAGAATTGGATGATACTGG |
| | TCTATCATTCATCCTTTCTGAAATTTT:ATTCAA |
| | TCTTTCCTGCTCCACTAGTTCCATCCCATTATCC |
| | CTGAAGTCTCTCCTAACTAAAAGAAAAAAAAAAA |
| | GTTCTTGACCCCACACATCTTCTTCATGGAATTA |
| | ATTTTTATCTCTAACATTAAATTTTTTCTCTTTT |
| | TTCAGCCTTCTTCTCCATACCTTCCTTATCAGCT |
| | TGCCCTTAAATAATGATTATCCCGGGTTCCAGTG |
| | TCCATTCTCTTCTCACTCTCTACCCTCTGAGTTT |
| | CTCACATATACAGGAACTCATATGTACCCCTGAG |
| | GGATTGGTTCCAGGACTCTGTTGGAAACCAAAAT |
| | CCACGAATGCTCAAGTCCCTTACATAAATGATGT |
| | ACTTATACACATCATCCCACATCTTTAAATTATC |
| | TCTAGATTACTTATAACACCTAATACAATGTAAA |
| | TGCTATGCAAATAGTTGTTGTATTGCTTTTTGTG |
| | TTTTTTTTAAATTGTTTCTTTGTTATCTTTTATT |
| | TTTCCCAAATTATTTCTGACCCAAAGTTGGCTGA |
| | ATCCATGGATCTGGAACCCATTAATATGGAGGGC |
| | TAACTGTATACCCAACTGCTTATGAGACCTCCAC |
| | CTGAACATTTTTGAGACATCTCAAGTCAACTAAT |
| | CATTTTTTTTTACAATTTTTCTAACCTTAGTACT |
| | TTTTTTTTTTTTTTTCCCTGAGACAGAGTCTTGC |
| | CCTGTCGCCTGGGTCAGAGTGCAGTGGCACGATC |
| | TCAGCTCACTGCAACTTCCACCTCCCAGGTTCAA |
| | GTGATTCTCCTGCCTCAGCCTCCCAAGTAGCTTT |
| | AAAGTATATGCAGAGTCTGATACCTTTTCTCTCC |
| | ACTTCTACCACCCGGGTCCAAGTGGCCTTTTTTC |
| | CTTGCCGGTGTTACTGCAGTAGCTCCCCTACTGC |
| | TCTCCCTGCTGTTGCCTGTGTTGCCTGCAGTCTT |
| | TCCAATGTTAGCAGTCAAAGTCATCTCTCAGAGC |
| | CAGAGCTGGATCATGTCCCTCCTCTGCTGAAAGC |
| | AGTTCGTGATTCATCCTCTTGAGGCAAAACCTCA |
| | GCCCTCACAGAAGTACCCTCTGCTCCAGCCTCCC |
| | CCATCCAGAATCTCATGGGACACCACACTTTGTC |
| | CTTGGTGTCCTTTAGATGCTCAGGTGCCAGTTCC |
| | CCTCTCAGTCTTCCACAGTTGCTCTGATCTCCTC |
| | AGACACTGTCAGGGCCTCGTAGGGCCTGCTGGGC |
| | TGACTCCTCAGTTCCTTCCCGATCATACATAACT |
| | ATCTTCCCTGTGAGGCAGTCCCTTACCACCCAAT |
| | TTAAAATTGCACCTCCTTCCTCCAACCGGTCCCT |
| | CTGCTGTGATTTGGTTTTTCTCTCTTACTTTCCT |
| | TTTAGTTATTTACTTATTTATTTTGTTTGACTCT |
| | CTCCACAAACAAAGCTTAATTAAGCTGCATGAAG |
| | GAATCGTTGTCCATAATGTTTACTGATGCATCTC |
| | CAGTGCCTAGGACAGTGTTTGACAGATGATAGGT |
| | GCACAATACATATTTGTTGCTTAAAATTATATGG |
| | GAGCTAATCATTGGAGGGAAAAATGTCTTCGGTT |
| | CTCTGTAGCACACTTTTTGGCAT |
| der(8) | GTTGCTGACATAGCTCTATCAGAAAGGTGCTCAG |
| | CGGCTGAATGAATGAATGAATGCGGTGGAGCATG |
| | AGGGCGCAGGCGCCATAGTGGGGAGGGGCAGGAG |
| | GGAATATTCTGCCGGCAGCCACAAGAATAGGAAA |
| | GAGAAATACTCTGTCCGAATGGTTTGTTTTTTTG |
| | TGTTGGTGGGATTGCTCAATGCATGGCTCTTCTT |
| | GATCAGTGAGAAATGTGATTGGAGAAAGACATTA |
| | GAGTTTTATAGCTGATGTTAAAAAATGAGAGCCG |
| | CCACATTGACACTATTTGCTTCCGGAGACCGATG |
| | AGAGTGAAGAACTAACTAACAGCTTTGGGCTAAA |
| | CTTTGGAGTGCATTTTTATTAATAAGGCTAGATG |
| | AACCATCGCTGGCTTTCCAGTTGTATGTATCCCT |
| | CATAGAATCAGACACCAACTCAGGGAGGGGCCTT |
| | TCCAGCACACAGAGGGCTCTGCCTTTTTTTTTTT |
| | TTTTTTTTGGGAGTCTTGCTTTGTCGCCCAGGCT |
| | GGAGTGCAGTGGCCGATCTCACCTCACTGCAACT |
| | CCGCCTGCTGGGTTCAAGGAATTCTGCATCAGCC |
| | TCCGGAGTAGCTGGGATTACAGGCACCTGCCACC |
| | ACGCCCAGCTAATTTTTGTGTTTTAGTAGAGACA |
| | GATTTTCACCATGTTGGCCAGGCTGGTCTCAAAC |
| | TCCTGATCTCAGGGGATCCGCCTGCCTTGGCCTC |
| | CTAAAATGCTGGGATTACAGGCGTGAGCCACCGC |
| | GCCCAGCCCCTTCCTCTTTTTGGCTTCCACTTGG |
| | CCAAAGGAGCTTCCCTTTGGGGCCGCGTCTGAGT |
| | CATGTTAGCCGCTGGTTCAAGGGAGCCATCTGAA |
| | ACTCCTTGGAGCAGGGTTTCTCCTGTATATGGCA |
| | ATACTCTACCTTAGAAAAACAAAACAAAACAAAA |
| | CAAAACGTCAAGTTACATGGTATAATGGAAAAAA |
| | ACCCTAAACTTGGGCTAGAAAATATAGGCCACTA |
| | TTTTGTTTCTAATACCACTATGTATATGGACAGA |
| | ACAGTTAACTGTCCACGCCCTGTGTCTTCCTTCG |
| | TGAAAGGTGGTGACACTGTACCTTCACAGCCTAC |
| | TTCATAGGACTTTGGTGTAAAATGAGACAGCAGA |
| | TGTTAAAGTACCTTCCGAGACCATTGTTTCCAGA |
| | ACCTTCATCACATTGTGGTATTAGAAACCTTGGA |
| | GTCCTCCTTGACTCTTTCACAAGCTACATTGAAT |
| | TTGTTCTCAAAGCCTCTGGCTCTGGGATTACAGG |
| | GGTGCCCCACCACCCTCAACTAGTTTTTGTATTT |
| | TTAGTAGAGACAAAGTTTCGCCATGTTGGCCAGG |
| | CTGGTCTCAAACTCCTGACCTAAGGTGATCCGCC |
| | TGCCTCGGCCTCCCAGTGTATTTTTTATCTCAGC |
| | TGGTAACACCAAAATTAATCAAAGTACTCAGTAA |
| | CTAGAGTGTCATGCTAGAATCTTCTGTTTCTCTC |
| | ACCATTGCTCTACAACCAATCAGAAATTCTTTCA |
| | ATTTACTTCTAAATTGCTTTATTTCACACCCTCA |
| | TGACCACTGTTTAGTTCAGGCTTTCAACTCTCTT |
| | GGATTATTTTAGATAGCTCTCCCTCCCCTAAGTG |
| | ATTTCCATAGTCTGGTCTCAATTTCTCAAATCCA |
| | TTATCTATATAACTGCTATAATTAACTTACTAAA |
| | AATAAATCAGATCATAACTTCTACTTTCTTACTT |
| | CAAACACTTCATTACCTCTTTAGTCACTTTCTAA |
| | AGCCTTTTATGATATGACTCATGACTCTGCCTTC |
| | CTCCCTCCAACCCCTAAACAAAACCAATGTTTCA |
| | AACTCTCTTGAACACACGCCTGTGCAATTTAAGG |
| | TAATTGTTTGTACACTTTATTCTTCTGCCTAGAA |
| | CAGCCTTCCCTTGATGTGCATACCAGATAACCTT |
| | AAAATGTAGCTCTGCTATCTCTTTCCTGAAACGT |
| | TTCTTTTTTGACTACTACTCCACAGTGATCCCCA |
| | CAAGGCAGAGTTAATTGCGTCTTCCTCTGGCCTA |
| | TCCCTGTACCTTATACTTGTTCCTAATACTCTAT |
| | TACATTACATAGTTGACCCTGCTATAAAAAAACT |
| | CAGAATTTGTGTTTTTCATAATGGTATTCATGAA |
| | TGATTCACGC |

The significance of the structural rearrangements disrupting this locus using copy-number variant data [11] was evaluated. The data were derived from 33,573 cases referred to a clinical diagnostic laboratory for array-based CGH testing (Signature Genomic Laboratories, PerkinElmer) and from 13,991 unaffected controls in previous genomewide association studies (Table 4).

TABLE 4
Gene-specific deletions in independent clinical samples

| | | Deletion | | | |
|---|---|---|---|---|---|
| ID | Chr | Start | Stop | Size | Indications for Study |
| 48921 | 8 | 61,786,528 | 61,900,621 | 114,093 | cardiac and renal anomalies |
| 69243 | 8 | 61,859,658 | 61,906,889 | 47,231 | multiple congenital anomalies, |
| | | | | | craniosynostosis, |
| | | | | | congenital heart defect, |
| | | | | | hypoparathydroidism, deafness |

Two gene-specific CHD7 deletions were identified among 33,573 subjects referred to clinical diagnostic laboratories for array CGH analyses. The indication for study in both subjects was consistent with features of CHARGE syndrome. No gene-specific alterations (deletion or duplication) were identified within LMBRD1 or among 13,991 control individuals.

### Results

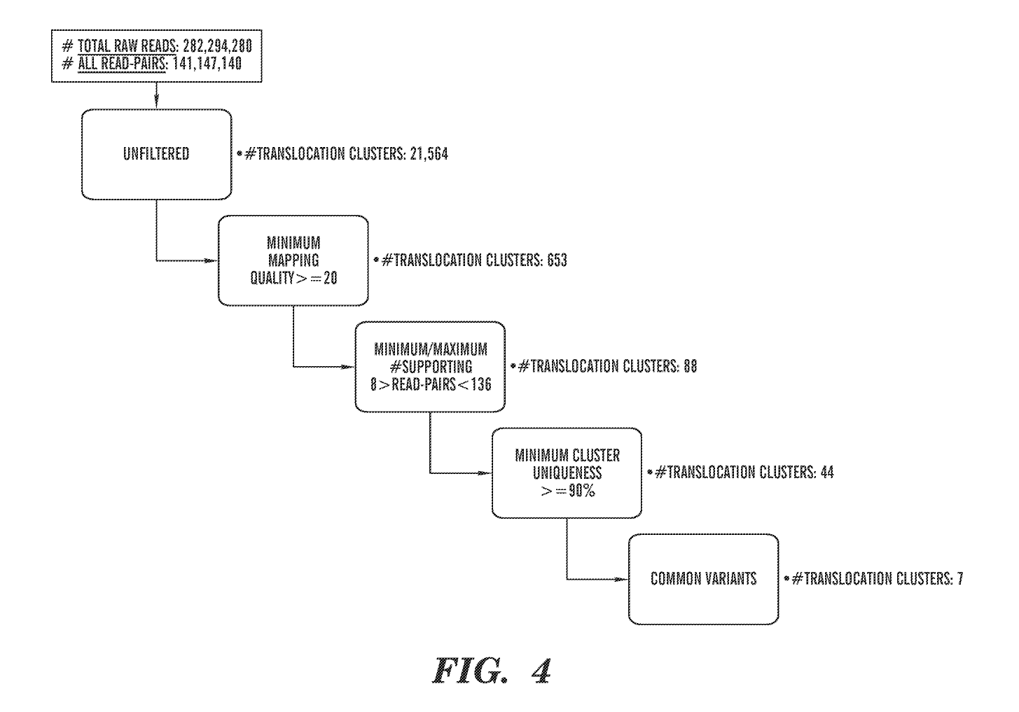

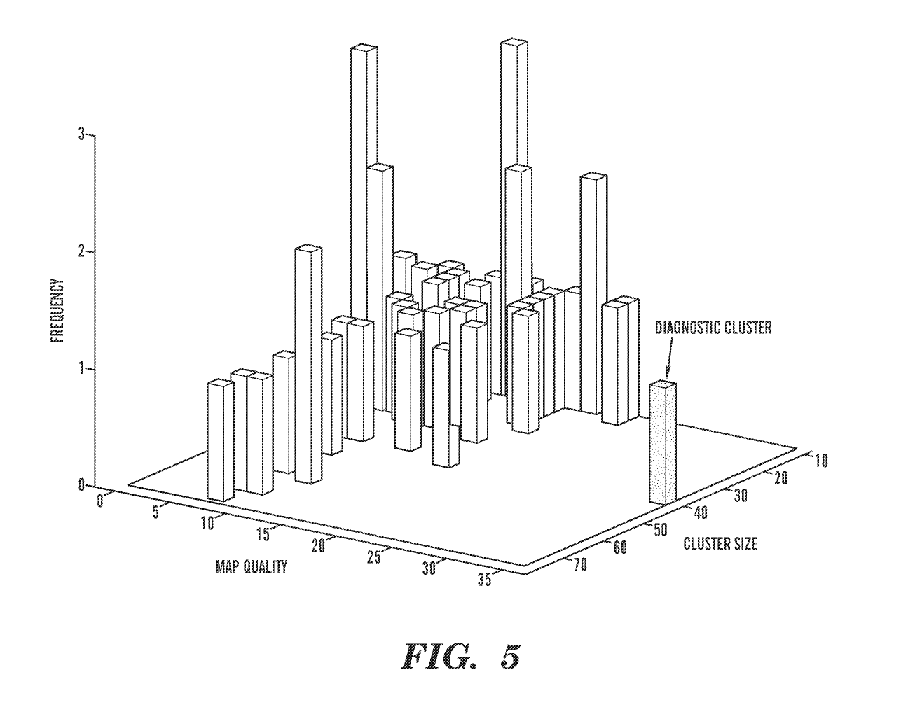

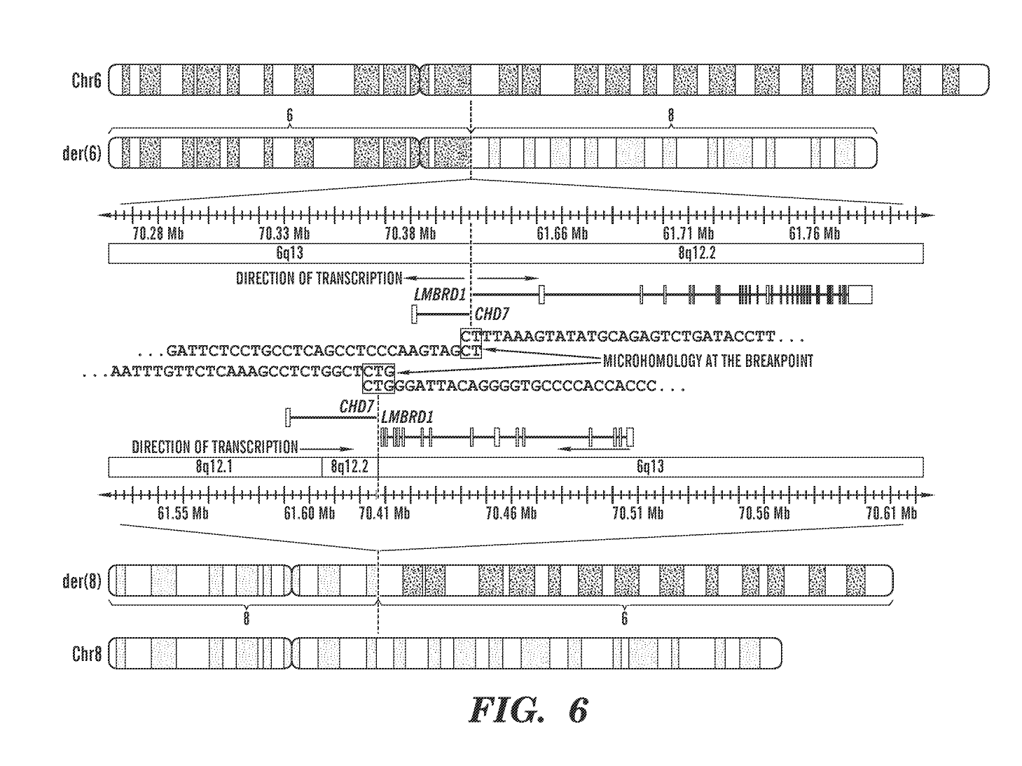

Large-insert, paired-end sequencing of DNA from cells in the amniotic fluid generated 282,294,280 individual reads (141 million pairs). Each aligned pair allowed assessment of a chromosomal region corresponding to the original fragment size (median, 1914 bp; standard deviation, 369 bp). Consequently, the inserts between aligned pairs covered each base in the genome 68 times on average, despite a mean coverage of only two reads spanning each nucleotide of the genome. One cluster of reads was identified with ends mapping to chromosomes 6 and 8 (FIG. 4). This cluster contained 35 read pairs with high mapping quality (FIG. 5). The translocation breakpoint in chromosome 8 directly disrupted CHD7, and the chromosome 6 breakpoint disrupted LMBRD1 (FIG. 6). The transcriptional orientation of each gene was incompatible with the generation of a fusion transcript involving CHD7 and LMBRD1. PCR amplification and capillary sequencing of the breakpoints resulted in a revised karyotype of 46,XY,t(6; 8)(q13; q12.2)dn.

About 94.7% of all reads aligned to human genome reference hg19. Insert sizes were tightly distributed around the 2 kb targeted selection. Over 99.9% of properly aligned pairs contained large inserts. The analyses showed 39 such candidate translocation clusters in the genome after filtering (representing just 0.00057% of all read pairs), of which only one candidate cluster mapped to chromosomes 6 and 8. PCR amplification and Sanger sequencing confirmed the predicted translocation event. The chromosome 6 breakpoint resulted in loss of a single base between the der(6) and der(8) chromosomes and chromosome 8 sequence was perfectly balanced. Microhomology was observed at each breakpoint, 2 bp in der(6) and 3 bp in der(8), resulting in a sequence based cytogenetic interpretation of der(6):6pter->70405867::chr8 61628671->qter with 2 bp breakpoint homology and der(8):8pter->61628669::chr6 70405868->qter with 3 bp homology (FIGS. 2A-2B and Table 3).

Point mutations of CHD7 cause the CHARGE syndrome, which has also been attributed to functional hemizygosity arising from deletion of one copy of the gene [12]. Copy-number variant data on more than 47,000 persons were analyzed, and two gene-specific deletions of CHD7, both of which were found in persons with features consistent with the CHARGE syndrome, were identified. There was no findings of LMBRD1-specific variations among cases referred to a clinical diagnostic laboratory for array CGH testing, or disruption of either locus among controls (Table 4). Taken together, these findings suggest that functional mutations and the disruption of a single copy of CHD7 by means of structural variation can cause the CHARGE syndrome.

### Discussion

The identification of a 46,XY,t(6; 8) (q13; q13)dn karyotype in a fetus was shown herein with an isolated heart defect at 18.8 weeks of gestation and additional abnormalities revealed on imaging studies performed throughout the third trimester (FIGS. 1A-1F). After delivery, the neonate received a clinical diagnosis of the CHARGE syndrome, a result that could not have been unequivocally diagnosed on the basis of ultrasonography, original karyotyping, or subsequent array-based CGH testing. Following an optimized 13-day protocol, large-insert sequencing of the prenatal DNA sample was used to identify precise translocation break-points that directly disrupted CHD7 at 8q12.2, a pathogenic locus in the CHARGE syndrome, 12 and LMBRD1 at 6q13, a pathogenic locus in a recessive disorder of vitamin B12 metabolism (cobalamin F type) 13 (FIG. 6). Accordingly, a pathogenic gene disruption was identified by sequencing the DNA obtained from a prenatal sample with a balanced translocation, providing a definitive sequence-based prenatal diagnosis that was consistent with the diagnosis based on postnatal clinical findings.

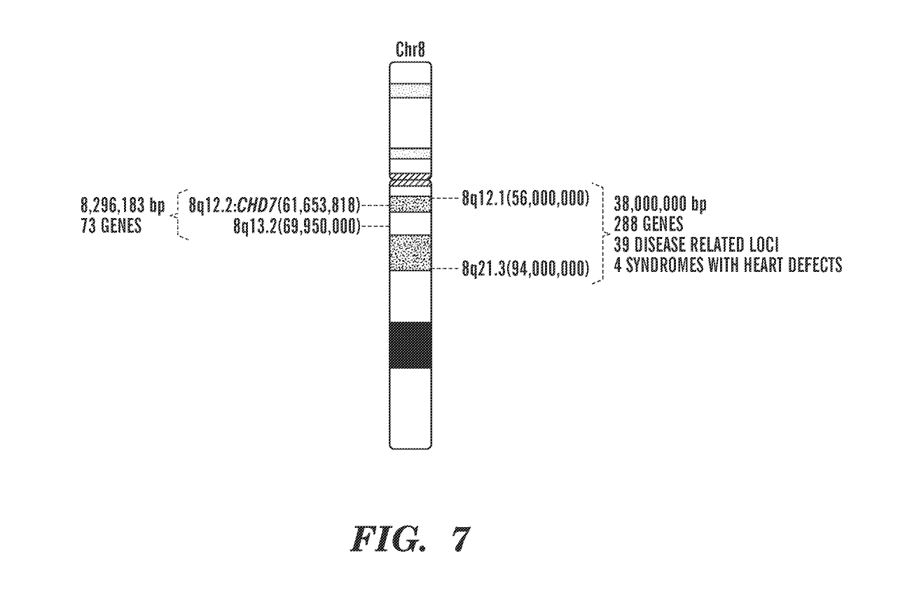

The findings presented herein indicate that innovations in genome sequencing aimed specifically at detecting structural variations can offer a rapid adjunct to cytogenetic techniques. Sequencing enables precise definition of individual disrupted genes, thereby adding to the information available for outcome prediction, medical planning, and genetic counseling. In the Example described herein, results obtained with cytogenetic testing and array-based CGH were consistent with a balanced de novo translocation, but these tests did not identify the gene or genes responsible either for the isolated cardiac defect or for the additional fetal abnormalities that were subsequently detected. Designation of the 8q13 breakpoint through karyotyping neither supported a prediction of a disruption in CHD7 at 8q12.2 nor provided sufficient resolution to consider specific genes in a differential diagnosis (FIG. 7). Indeed, were GTG-banded breakpoints to be misinterpreted by a visible band in each direction (which is not an uncommon occurrence according to sequencing of such balanced rearrangements [8, 11]), this would entail consideration, on chromosome 8 alone, of approximately 38 Mb of DNA containing 288 potential phenotype-contributing genes, of which 39 have been associated with disease, according to the OMIM database, and at least 4 have been associated with cardiac defects. In addition, rearrangements appearing to be balanced at karyotypic resolution can be complex at nucleotide resolution (with complex rearrangements accounting for approximately 20% of all events) [8]. In the Example described herein, sequence-based revision of the karyotype permitted a definitive description of the causal syndromic locus. Such diagnostic precision and consequent phenotypic prediction cannot currently be obtained with the use of other methods, and the results were obtained within a time frame similar to that required for conventional prenatal cytogenetic methods.

The CHARGE syndrome is a rare, usually sporadic disease that may include cranial-nerve abnormalities and tracheoesophageal fistula in addition to other known clinical features [14, 15]. Previous studies have implicated CHD7 alterations in 90% of patients meeting the diagnostic criteria for the CHARGE syndrome [12]. CHD7 is a highly conserved member of the chromo-domain helicase family; it alters gene expression by remodeling chromatin [16]. CHD7 mutations thus have potentially wide-ranging phenotypic effects. Disruption of CHD7 represents a strong genetic risk factor for the CHARGE syndrome, although not all disruptions of CHD7 are fully penetrant, since deletions affecting a portion of the upstream or coding region of CHD7 have been identified in phenotypically normal persons of Asian and African ancestry [17, 18]. Nonetheless, if a CHD7 mutation is detected, clinical follow-up and genetic counseling are recommended [18].

The chromosome 6 breakpoint disrupted LMBRD1, which encodes a lysosomal membrane protein involved in the transport and metabolism of cobalamin. Frameshift mutations leading to loss of function of LMBRD1 are associated with the recessive disorder methylmalonic aciduria and homocystinuria (cobalamin F type) (OMIM number, 277380) [13]. Disruption of a single copy of the locus is unlikely to result in the disorder, and the postnatal metabolic workup of the present case ruled out a metabolic syndrome.

Delineation of a CHD7 disruption and consequent diagnosis of the CHARGE syndrome would probably have influenced genetic counseling, subsequent discussions of management of the pregnancy, and preparation of the health care team and parents for the possibility of multiple life-threatening medical conditions requiring immediate management of breathing and feeding difficulties on delivery [19]. If a clearly predictive causal locus was not detected, the rearrangement for its likelihood of representing a benign alteration or its designation as a variant of unknown significance could be assessed, using analyses of the clinical diagnostic and population-based copy-number variant data, along with available findings of standing genetic variation from exome-sequencing studies, resources such as the 1000 Genomes Project, and genomewide association studies in clinical cohorts and controls. Although the merits, limitations, and interpretation of such additional data sets warrant careful discussion and appropriate caution in view of limited understanding of the functional consequences of disrupting specific sequences in the human genome, medical decisions are usually considered based on the presence of the chromosome rearrangement, without additional predictive information. At best, a secondary array-based CGH test is performed, which in this subject and most subjects with apparently balanced abnormalities will yield a normal result. Our study thus shows the predictive power of pangenomic paired-end sequencing and points toward the complexity of interpretation likely to confront the enterprise of ultra-high-resolution diagnostics.

The strategy and approach presented herein, when used in the prenatal setting, can detect genomic alterations that may change the obstetrical course and outcome, providing a basis for decisions regarding termination, fetal therapy, mode of delivery, and postnatal referral to a tertiary-care center with advanced expertise in management.

### Example 2

### Example Design of Large-Insert Jumping Libraries for Structural Variant Detection Using Illumina Sequencing

Balanced chromosomal rearrangements are not routinely detected by microarray and localization of altered regions by karyotype is imprecise. The degree of resolution that can be obtained through next generation technologies enables elucidation of precise breakpoints and facilitates the discovery of numerous pathogenic loci in human disease and congenital anomalies. Described in this Example is an exemplary protocol to generate one type of large-insert “jumping library” for multiplexed sequencing using Illumina sequencing technology. This approach allows for cost-efficient multiplexing of samples and derives a very high yield of fragments with large insets, or ‘jumping’ fragments.

There have been several different approaches described to perform massively parallel sequencing of large fragments on multiple platforms. These methods have been referred to under various terms, such as large-insert sequencing, mate-pair sequencing, and jumping libraries, which all represent similar approaches to sequencing large genomic inserts. A variation of the jumping library method applied in next-generation sequencing was previously reported by Illumina in which circularization is achieved by blunt end ligation (Korbel et al., 2007) (Illumina Inc., San Diego, Calif.). Applied Biosystems developed a derivation of this protocol using an internal adaptor for circularization and restriction digestion to fragment the circle, followed by sample preparation for ABI SOLiD sequencing (additional information can be assessed at http://tools.invitrogen.com/content/sfs/manuals/SOLiD4_Library_Preparation_man.pdf. Applied Biosystems SOLiD™ 4 System Library Preparation Guide, 2010). The protocol described below is an adaptation of the mate-pair library protocol used with the Applied Biosystems SOLiD sequencing platform (Applied Biosystems SOLiD™ 4 System) but adapted for Illumina's sequencing by synthesis technology (Talkowski et al., Am J Hum Genet. 2011 Apr. 8; 88(4):469-81). The approach described herein is efficient in terms of lowest cost and highest proportion of large inserts generated. In some embodiments, there can be a tradeoff in terms of the length of the sequence fragments generated. Multiplexing can be accomplished by using individual Y shaped adapters, which contain Illumina compatible barcoding sequence on one strand.

In some embodiments, the method described herein can be used for creating DNA libraries with large genomic inserts for at least about 25 base pairs paired-end Illumina platform sequencing (e.g. suitable for 25 cycles of next generation sequencing). The method derives short fragments appropriate for massively parallel sequencing, for example, on an Illumina™ platform (Illumina Inc., San Diego, Calif.) that are separated by large genomic inserts of a user-selected size.

In overview, genomic DNA is sheared to a targeted insert size, circularized, then fragmented with the circularization junctions retained, thereby generating short genomic fragments in which the ends are separated by the size of the circle. This method enables massively parallel sequencing of the ends of the fragments and leveraging the size of the insert into information about the assembly of the genome. The methods described herein can be used to, for example, identify structural variation in the genome, including the complex reorganization of ‘shattered’ chromosomal segments that has been defined as chromothripsis, to delineate transgenic integration sites, to identify genes disrupted by balanced structural variation that contribute to human disease, or to characterize structural variation in prenatal diagnostic testing.

Exemplary materials and equipment used in the protocol described below are shown below. Modifications to the protocol, e.g., using other functionally equivalent materials or equipment, within one of skill in the art are also within the scope of the inventions described herein.

### Materials

| | Vendor/cat. no. |
|---|---|
| Reagents | |
| 5-10 ug genomic DNA sample | sample |
| 1X TE Buffer | 12090-015 |
| QIAquick PCR Purification Kit | 28104 or 28106 |
| End-It DNA End-Repair Kit | Epicentre, ER81050 |
| RNase-Free Duplex Buffer | IDT |
| Cap adapter oligo duplex | Described below |
| Quick ligation kit | NEB, M2200S or M2200L |
| UltraPure Agarose | Life Technologies, 16500-500 |
| Ethidium Bromide | Sigma, E1510-10ML |
| 1 kb + DNA ladder | Life Technologies, 10787-018 |
| QIAquick Gel Extraction Kit | 28704 or 28706 |
| Quant-iT PicoGreen dsDNA Assay Kit | Life Technologies, P7589 |
| internal circularization adapter duplex | Described below |
| Plasmid-safe DNASE kit | Epicentre, E3105K |
| EcoP15I restriction enzyme | NEB, R0646L |
| BSA 100X | NEB. B9000S |
| 10 mM ATP (contained in NEB EcoP15I package) | NEB |
| NEB Buffer 3 (contained in NEB EcoP15I package) | NEB |
| Sinefungin, 10 mM | Millipore, 567051-2MG |
| Klenow DNA polymerase I (lg) fragment | NEB, M0210S |
| Nuclease-free water | Ambion, AM9930 |
| Sodium Chloride(5M) | Boston BioProducts, BM-244 |
| Tris-HCL Buffer (1M, pH 7.5) | Boston BioProducts, BM-315 |
| EDTA (0.5M, pH 8.0) | Boston BioProducts, BM-150 |
| Dynabeads MyOne Streptavidin C1 | 65001 |
| NEBNext dA Tailing Module | NEB, E6053L |
| barcoded Y adapter duplex | Described below |
| Phusion High-Fidelity PCR MM w/HF Buffer | NEB, M0531S |
| Agilent 1000 kit | 5067-4626 |
| Equipment | |
| NanoDrop spectrophotometer | ND1000 |
| Covaris Focused-ultrasonicator (S-Series or E-Series) | Covaris |
| Covaris miniTUBES(blue) for 3 kb shearing | 520065 |
| Centrifuge | Eppendorf 5804 |
| Gel electrophoresis apparatus | BIO-RAD |
| Vacuum Manifold (optional) | Qiagen |
| ThermoCycler | BIO-RAD C1000 |
| Heat Block or incubator (37 C., 65 C.) | eppendorf |
| Magnetic Rack | Invitrogen CS15000 |
| Agilent 2100 Bioanalyzer | Agilent 2100 |
| | or equivalent QC |
| | method |

Provided below is an example protocol to generate a large-insert jumping library for structural variant detection using sequencing by synthesis technology (e.g., Illumina™platform). While human genomic DNA is used in the following protocol, genomic DNA from other species can also be used.

### A. Fragmentation of Human Genomic DNA

Shear approximately 5-10 ug of high quality human genomic DNA to a target size of 3 kb. For example, a Covaris S2 instrument can be used to shear the genomic DNA using Covaris' nominal shear protocol.
Clean up the fragmented genomic DNA, e.g., by following the manufacturer's instructions for the Qiagen PCR Purification Kit (or, where applicable, Qiagen Gel Purification) for each reaction cleanup. In one embodiment, the elution volume for this step is 35 ul per 5 ug starting material.

It should be noted that the initial DNA sample should be of high-quality. Preferably, at least 5 ug or more in quantity is desired. Poor quality, degraded DNA and/or insufficient starting material can result in both poor library yield and high duplication rates. The following steps are optimized for 10 ug of starting material. Reaction volumes can be scaled proportionally for different initial quantity.

### B. End Repair of Sheared DNA

End repair the sheared DNA fragments from step (A) using blunt ending method. For example, the Epicentre's EndIt End Repair kit (or equivalent blunt ending method suitable for subsequent ligation) can be used herein. In one embodiment, one reaction volume (50 ul) is used per 5 ug starting material. Qiagen column purification, 50 ul elution volume.

### C. Cap Adapter Ligation

Determine the concentration of sheared, end-repaired DNA, for example, using a Nanodrop instrument. Determine the volume of duplexed 50 uM cap adapter required. In some embodiments, an adapter:DNA fragment ratio of ˜10:1 can be used.

The cap adapters can comprise an endonuclease recognition site. While various endonuclease recognition sites can be selected, an exemplary endonuclease recognition site used herein comprises an EcoP15I recognition site, and example sequences of EcoP15I cap adapters (initial circularization adapters) are shown below:

### EcoP15I Cap Adapter/Initial Circularization Adapter (Duplex, 50 uM) (5′->3′)

Cap_adapter_1:
/5Phos/ACAGCAG
Cap_adapter_2:
/5Phos/CTGCTGTAC

Initial preparation of the capping oligos, as well as all other double stranded adapters used within this protocol, can be achieved by resuspending both strands at the desired concentration in duplexing buffer (IDT RNase-Free Duplex Buffer). Oligos can be briefly denatured by heating to 94° C. for about 2 minutes, and then allowed to cool to form double-stranded adapters. Once prepared, duplexes may be stored at −20° C. until needed.
Ligate the cap adapters to the sheared, end-repaired DNA. In some embodiments, the NEB Quick Ligation Kit reagents and protocol can be used for such purpose.

### D. Gel Size Selection

Prepare a 0.8-1% agarose (EtBr, 2.5 ul per 150 ml) gel to use for more specific size selection.
Add 10 ul 6× loading dye to the sample and load the entire product across two lanes. The volume of loading dye loaded into a single lane can vary proportionally with the amount of starting material. Run the gel until a bromophenol dye indicator has migrated approximately 2-3 cm. Running the gel longer than about 2-3 cm migration may result in the need to use more than one qiaquick column due to agarose mass.
Extract the product from the gel based on a user-selected size and purify the extracted product. In some embodiments, the selected size for genomic DNA fragments can have a range of about 2.5-6 kb. For example, Qiagen reagents, e.g., 50 ul elution volume, can be used for product purification.

### E. Circularization

Determine the volume of duplexed 2 uM internal adapter, e.g., using a NanoDrop or PicoGreen measurements. In some embodiments, an internal adapter:DNA fragment ratio of ˜3:1 can be used. An exemplary yield at this stage can be around 1 ug of DNA (per 5 ug starting material), but libraries can also be prepared with ˜0.5 ug of DNA or less.

If NanoDrop measurements are used, a strong solvent absorption peak<230 nm can be present. Comparison with PicoGreen measurements has determined that readings remain adequately reliable for this step. In some embodiments, PicoGreen measurement can be used if the solvent absorption maximum occurs at a wavelength greater than 230 nm.
Circularize the gel purified product from step (D) using the internal adapter, which contains a biotinylated thymine that can be used later for selection of DNA fragments comprising the circularization junction. The ligated cap adapters of step (C) are designed to contain overhangs which are complementary to the overhangs of the internal adapter. In some embodiments, the cap adapters of step (C) can be designed to contain AC overhangs, which are complementary to the overhangs of the internal adapter. Example sequences of the internal adaptors (circularization adaptors) are shown below:

Circularization Adapter (Duplex, 2 uM)(5′->3′):

Internal_1A:
/5Phos/CGT TC/iBiodT/CCG T
Internal_2A:
/5Phos/GGA GAA CGG T

In order to help reduce the number of chimeric circularizations, the DNA concentration can be diluted, e.g., as low as about 2 ng/ul or lower.

An example reaction sample for circularization is shown below. For example, NEB quick ligation reagents can be used to set up the following 500 ul reaction per 1 ug DNA (scale reaction volumes as needed):

| | |
|---|---|
| DNA(1 ug) | 50 ul |
| NEB quick ligase buffer: | 250 ul |
| H₂O: | 187 ul |
| Internal adaptors (2 uM) | 0.76 ul |
| NEB ligase: | 12 ul |

Qiagen column purification, 60 ul elution volume.

### F. DNase Treatment

Remove non-circularized product by treating the circularized product mixture with a DNase. For example, Epicentre's Plasmid Safe DNase reagents can be used to remove non-circularized products. Qiagen column purification, 30 ul elution volume.

### G. Fragmentation of Circularized Products

The circularized products can be fragmented to produce linear DNA fragments by endonuclease digestion or random shearing (e.g., random acoustic shearing). In this Example, the circularized products are fragmented by endonuclease digestion. The following example reaction is an illustration of using an EcoP15I endonuclease to digest overnight (min 12 hour) at 37° C. Depending on the endonuclease recognition site present in the cap adaptors of step (C), different endonuclease enzymes can be used instead.

EcoP15I Digest (100 ul Reaction)

| | |
|---|---|
| Circularized DNA | 30 ul |
| 10X NEB Buffer3 | 10 ul |
| 10X BSA | 10 ul |
| 10X ATP(25 nM) | 20 ul |
| H2O | 23 ul |
| Sinefungin (10 mM) | 1 ul |
| EcoP15I(10 U/ul) | 6 ul |

Following the incubation (e.g., of about 12 hours), the endonuclease enzymes can be heat-inactivated. For example, add 2 uL 25 mM ATP (NEB) and 0.5 uL EcoP15I enzyme and incubate for an additional hour, then incubate at 65 C for 20 minutes to heat inactivate enzymes.

### H. End Repair of Digested DNA

End repair the digested DNA of step (G), e.g. by adding about 1.5 uL 25 mM (each) dNTP mix and 1 ul Klenow (large) fragment to the reaction mixture. Allow the end repair reaction to proceed for 40 min at room temperature, then heat inactivate again by incubating at 65 C for another 20 minutes. Cool reactions on ice for 5 minutes.

### I. Streptavidin Bead Binding

Wash 35 ul of streptavidin-coated beads (e.g., MyOne C1 beads) for each library preparation with bead wash buffer as prepared below. As long as the volume permits, the total required volume of beads can be washed in a single tube. Add 500 ul wash buffer to the beads, vortex the tube briefly to homogenize the mixture, and then quick centrifuge to remove any liquid from the tube cap. Use a magnetic rack to isolate the beads. Repeat, e.g., for a total of three washes.
Resuspend the washed streptavidin-coated beads in their original volume of 1× binding buffer (1 part binding buffer as prepared below, 1 part nuclease free water).
Add 105 ul binding buffer and 35 ul washed bead solution to each ˜105 ul DNA sample. Bind for approximately 30 minutes at room temperature with periodic mixing.
After binding, wash again three times with 500 ul wash buffer. Wash buffer should be removed as much as possible in this and all other final wash steps, as residual wash solution can interfere with subsequent enzymatic reactions. It can be helpful to quickly spin the tubes after the final wash to draw remaining liquid to the bottom for easier removal of residual wash solution.

An example preparation of streptavidin binding buffers and wash buffers is shown below. Volumes of reagents can be scaled as needed. Buffers can be stored at room temperature for three months.

| | | |
|---|---|---|
| Tris-HCL 1M PH-7.5 | 60 | ul |
| NaCl 5M | 2.4 | ml |
| EDTA 0.5M | 12 | ul |

for wash buffer: bring volume to 12 ml with nuclease free water and add 1 ul TWEEN20.
for binding buffer: bring volume to 6 ml with nuclease free water.

### J. dA Tailing of DNA (on Streptavidin-Coated Beads)

Add a single A base to the end repaired DNA. For example, NEB dA tailing module can be used following the manufacturer's protocol for 50 ul reactions. After the reactions are complete, wash the beads again three times with 500 ul wash buffer. A final quick centrifugation can be performed to remove remnants of wash buffer.

### K. Adapter Ligation (on Streptavidin-Coated Beads)

Ligate custom barcode adapters to the DNA fragments that are bound to the streptavidin-coated beads, e.g., using NEB Quick Ligation Kit. For example, ˜0.34 uL 15 uM duplexed barcode adapter can be used for each 1 ug circularized DNA (as measured in step (E)).

### Example Sequences of Barcoded Adapters are Shown Below (Duplex, 15 uM) (5′->3′):

UniversalMPX:
ACACTCTTTCCCTACACGACGCTCTTCCGATC*T
MPX_index##:
/5Phos/GATCGGAAGAGCACACGTCTGAACTCCAGTCAC(+6BPindex)
*Phosphorothioate (S-oligos) are used to help
prevent degradation

After the reaction is complete, wash beads again three times with 500 ul wash buffer, and once with 500 ul elution buffer. A final quick centrifugation can be used to remove remnants of buffer. Resuspend cleaned reactions in 30 ul elution buffer.

### L. PCR Amplification (on Streptavidin-Coated Beads)

After barcode adapters are ligated and reactions washed, libraries are amplified by PCR using a universal forward primer and a reverse primer dependent upon the specific barcode used. In this Example, three PCR reactions are run per library in order to reduce random amplification bias. An initial cleanup prior to final gel purification concentrates the sample for gel loading and also removes excess primers and dNTPs from solution. For each library, prepare three 50 ul PCR reactions using the reagents and ratios indicated below.

### Exemplary Materials Needed for PCR Amplification

Barcode-ligated DNA sample (on beads, resuspended in 30 ul EB)
Phusion High-Fidelity PCR MM w/HF Buffer (NEB, M0531S)
Univ_PCR forward primer (25 uM)
Index compatible PCR reverse primer (25 uM)
Nuclease free H2O

### Example PCR Reagents for Three 50 ul Reactions:

| | |
|---|---|
| DNA(on beads) | 30 ul |
| Primer 1.0 (25 uM) | 1 ul |
| Index Primer (25 uM) | 1 ul |
| 2X Phusion mix | 75 ul |
| Nuclease free water | 43 uL |

### Example Sequences of PCR Primers (25 uM) are Shown Below (5′->3′):

Univ_PCR:
AATGATACGGCGACCACCGAGATCTACACTCTTTCCCTACACGACGCTC
TTCCGATC*T
Rev_index#:
CAAGCAGAAGACGGCATACGAGAT(+6BPprimerindex**)GTGACT
GGAGTTC
*Phosphorothioate (S-oligos) are used to help
prevent degradation
**rev primer index is the reverse complement of
the barcode adapter index

The PCR setup and protocol for library amplification are shown below for illustrative purposes and are not construed to be limiting. The PCR setup and protocol for library amplification below can be optimized to suit a user's need.

Example PCR Protocol:

98° C. 30 seconds
98° C. 10 seconds
65° C. 30 seconds
72° C. 30 seconds X 8-12 total cycles
72° C. 5 min, 10 C. hold

### M. Gel Purification of PCR Products

Following amplification, use a magnetic stand to separate post-PCR solution from the beads and purify, e.g., on one Qiagen PCR purification column, 30 ul elution volume. Load the entire elution volume on a single lane of 2% agarose (EtBr 2.5 ul/150 ml gel) gel. Some variation in yield between libraries amplified with the same number of cycles is normal, but product should be clearly visible as a distinct ˜200 bp band. Extract this product using Qiagen gel purification reagents. Elute with 17 ul elution volume and add 1.5 ul 1% TWEEN20.

If peaks lower than ˜200 BP are present, run the gel longer in order to avoid possible contamination with dimer during gel extraction.

### N. Quality Control of Final Product

Assess gel purified products using quantitative PCR, e.g., Agilent Bioanalyzer 2100 or Agilent TapeStation 2200 methods.

The example protocol described above can be used to prepare DNA libraries suitable for at least about 25 cycles or more of next generation sequencing. This method, which is compatible with sequencing by synthesis technology, e.g., using the Illumina platform (Illumina Inc., San Diego, Calif.), can result in libraries comprising short DNA fragments that represent junctions formed from the circularization of larger (e.g., ˜3 kb or larger) genomic fragments. The use of short fragments derived from long genomic inserts allows higher effective genomic coverage while minimizing the cost of whole-genome sequence coverage, and this cost savings will scale with future decreases in sequencing costs.

In this Example, the protocol described herein can be used to produce Illumina adapter ligated, small DNA fragments in which the ends are separated by the initial user-selected fragment size (e.g., ˜3 kb). Paired-end sequencing can yield predictable ‘jumps’ in the genome following alignment for a high proportion (>99% in our previous studies) of fragments sequenced, enabling routine delineation of structural variation or genome assembly.

Parameter Consideration:

Both the quantity and quality of the starting material should be assessed, as these factors can significantly influence the quality of finished libraries. Degraded DNA will result in both poor library yield and high duplication rates. For example, the DNA quality can be determined by a genomic sample (˜100-200 ng) on a 1% agarose gel to determine DNA quality. Genomic DNA should be visualized as a distinct high molecular weight band with little to no visible smearing present. PicoGreen, or another method more specific to doublestranded DNA, can be used for initial quantification. Absorption measurements at a wavelength of about 260 nm can be less accurate for measuring the concentration of intact, dsDNA. In addition to initial DNA quantity, other factors that influence the quality of a library include, but are not limited to, reaction efficiency, number of PCR cycles, and the final gel purification. Residual wash buffer, used in steps (I)-(K), can interfere with the enzymatic reactions associated with these steps. Thus, residual wash buffer should be removed for maximum reaction efficiency.

The number of PCR cycles in the final amplification can influence the degree of amplification bias. Using fewer cycles can result in libraries with less bias, however too few cycles may not provide DNA in sufficient quantity. DNA yield following the initial gel size selection can provide a good way to initially estimate the number of PCR cycles to use. For example, if DNA concentration following the initial gel size selection is roughly 1 ug or higher, then 8-10 amplification cycles may be used to produce amplified products in sufficient quantity.

The final purification steps (M) are performed for removing traces of primer and/or primer dimer, which can interfere with sequencing, from the final libraries. The final gel should be run long enough to cleanly isolate the ˜200 bp band during gel extraction from dimer bands (if present) that generally run at ˜130 bp. For example, the final products should run as a clearly visible, distinct ˜200 bp bands on an agarose gel and/or visualize as sharp peaks on Bioanalyzer or TapeStation traces. Final expected concentration can vary depending on starting quantity, reaction efficiencies, and/or PCR cycles used, but is generally between 5-100 nM.

Time Considerations:

Total protocol time can vary, for example, depending upon the number of samples being prepared and equipment available. For example, shearing time using Covaris 3 kb nominal protocol is 10 minutes per sample. The time required for size selection and final gel purification also varies, since the number of samples that can be run on a single gel is limited. The amount of time needed to prepare, purify and wash reaction samples can also influence the overall schedule. The EcoP15I digest is completed overnight, so it is practical to time other preceding reactions around this step. In general, a small number of samples can be prepared in two days or less. For a larger number of samples, it can take longer to prepare the library (e.g., 3 days or more). In one embodiment, eight samples can be simultaneously prepared using the example protocol described herein, e.g., in about three days or less.

### Example 3

### Exemplary Bioinformatics for Whole-Genome Analysis

The following computational modules of the whole-genome analysis can be applied to any collection of sequence data. In some embodiments, the sequence data are obtained from large-insert jumping libraries. Each module or sub-modules below can be applied alone, or in combinations with other modules described below or with functionally-equivalent algorithms known in the art, for the whole-genome analysis.

### Module I: Computational Processing and Alignment

Raw sequencing reads can be collected into two paired files in FASTQ text format. Cock et al., Nucleic Acids Res. 2010; 38(6): 1767-71. For example, read-name (e.g., but not limited to, instrument name, flow cell lane, cluster tile coordinates), DNA sequence string, and base-calling quality information (in ASCII-printable characters) are stored for read-pairs generated by paired-end sequencing. In some embodiments, the raw read-pairs are in an outward-facing (or “reverse-forward”, RF) orientation to one another at this stage.
The raw read-pairs are then pre-processed as follows:

### Sub-Module for Pre-Processing of Read-Pairs.

a. Encoded base-quality strings can be converted from next-generation sequencing data (e.g., Solexa/Illumina (Illumina 1.3+, various ASCII encodings)) to Sanger (PHRED+33 offset) ASCII format for compatibility with downstream tools.
b. Read-pairs can be computationally inverted to an inward-facing (or “forward-reverse”, FR) orientation for compatibility with downstream alignment tools.
c. With provided base-quality information, reads can be trimmed to remove poor-quality bases. For example, the poor-quality bases can be removed in an iterative, windowed fashion using an open-source tool “sickle,” which can be accessed at https://github.com/najoshi/sickle (default parameters).
d. Optionally, read-pairs can also be trimmed when there is prior knowledge of adapter and barcode contamination, e.g., using an open-source tool “TrimGalore” v0.3.2 (“Trim Galore” Babraham Bioinformatics, Babraham Institute, which can be assessed at http://www.bioinformatics.babraham.ac.uk/projects/trim galore/), for example, with the “—paired” to process read-pairs, “—length 19” parameter to discard any trimmed read shorter than 19 bp, and the “—retain_unpaired” option to keep unpaired single ends with a mate that has been trimmed too short.
e. Overall quality of the library preparation, including molecular duplication rate as well as per-base quality in all reads, can be assessed, e.g., using an open-source tool “FASTQC” v0.10.1 [4]. This is a checkpoint before proceeding with alignment.
f. If the checkpoint passed, pre-processed reads can be split into chunks of 10 million reads to achieve alignment parallelism.
Read-pair chunks can be computationally aligned to a genome reference assembly in a parallelized manner using an alignment tool known in the art. In some embodiments, the genome reference assembly for alignment analysis can be a human genome reference assembly hg19 (build GRCh37 from Genome Reference Consortium), sequence data of which can be accessed at http://hgdownload.cse.ucsc.edu/goldenPath/hg19/bigZips/. In some embodiments, Wheeler short read alignment tool “bwa” v0.5.9+, e.g., using the ‘aln’ command (default parameters) followed by the ‘sampe’ command, can be used to computationally align the read-pair chunks to a genome reference assembly. Li et al. Bioinformatics. 2009; 25(14):1754-60. The latter can include the ‘-s’ option to disable local Smith-Waterman alignment with regards to unmapped mates when considering 25 bp chimeric read-pair alignments.
Individual parallel alignment outputs can be merged into a single binarized and compressed version of an open-source file format or any other compatible format. An exemplary open-source file format is SAM file format (Li et al., Bioinformatics. 2009; 25(16):2078-9). Accordingly, in some embodiments, the individual parallel alignment outputs can be merged into a single binarized and compressed version of the open-source SAM file format (herein, this binarized version referred to as ‘BAM’) using the command “samtools merge.”
The read-pair alignments are then post-processed as follows:

### Sub-Module for Post-Processing of Read-Pair Alignments

i. PCR and/or alignment duplicate read-pair alignments from the binarized and compressed file comprising individual parallel alignment outputs (e.g., BAM file) can be filtered, for example, using an open source tool “picard tools” v1.102+ with the command “MarkDuplicates” omitting the “REMOVE DUPLICATES” Boolean flag.
ii. Marked duplicates can be removed, for example, using a custom Python script “get_duplicates.py” which separates the above marked duplicate reads or read-pairs from the non-duplicate read-pairs for further processing. Orphan, non-duplicate reads with duplicate mates can be tracked and stored. The resulting file is coordinate sorted, e.g., using the ‘SortSam’ command of picard tools with the ‘SORT_ORDER=coordinate’ option set.
iii. Each alignment in the binarized and compressed file comprising individual parallel alignment outputs (e.g., BAM file) can be further locally realigned for highly accurate indel and/or SNP downstream analysis capability. In some embodiments, each alignment can be further locally realigned using the Genome Analysis Toolkit (gatk) with the command ‘IndelRealigner’ and using the 1000 genomes datasets for reference sites of variation. Mckenna et al. Genome Res. 2010; 20(9):1297-303.
iv. Each sequence alignment's base quality in the binarized and compressed file comprising individual parallel alignment outputs (e.g., BAM file) can be further recalibrated, for example, using the gatk ‘BaseRecalibrator’ command with dbSNP and 1000 genomes datasets for reference sites. Mckenna et al. Genome Res. 2010; 20(9):1297-303.
v. The cleaned, analysis-ready file comprising individual parallel alignment outputs (e.g., BAM file) can be name and coordinate sorted, for example, with picard tools ‘SortSam’ with ‘SORT_ORDER=queryname’ and ‘SORT_ORDER=coordinate’ options set, respectively.

### Module II: Computational Analysis

Cleaned, analysis-ready, name-sorted files comprising individual parallel alignment outputs (e.g., BAM files) can be fed through a custom program, which can be designed to measure numerous statistics as well as categorize anomalous read-pairs found in the BAM file. An exemplary program for such purpose can be a custom C++ program-BamStat v0.2.0.
   The library statistics can be calculated using the selected program. In some embodiments, the library statistics can be calculated from the BAM by BamStat v0.2.0. Examples of the library statistics include, but are not limited to the following:
Overall measurements: Total Reads, Mapped Reads, Unmapped Reads, Mapped Read-pairs, Proper pairs (read-pairs within expected insert size of library, dynamically assigned by the selected alignment tool), and number of read-pairs with both ends mapped. For example, the read-pairs within expected insert size of library can be computed or assigned by the alignment tool BWA. Information about methods for estimating insert size distribution by BWA can be assessable online at http://bio-bwa.sourceforge.net/bwa.shtml#7.
Measurements about anomalous data: Summary of read-pairs with improper orientations (RR, RF, FR, FF) and read-pairs with neither end mapped, or one-end mapped.
Measurements about potential structural variants: Putative deletion pairs, insertion pairs, inversion pairs, and translocation pairs.
Insert size measurements: For FR and RF read-pairs, mean and median insert sizes, as well as standard deviation and median absolute deviation, respectively, of all proper-pairs found in the file.

All read-pairs with both ends mapped are then categorized by variant types or structural variant types. Examples of variant types or structural variant types include, but are not limited to, the following:
F=“Forward Strand”
R=“Reverse Strand”
Inversion-pairs: Read-pairs where the same DNA strand orientation on both sides indicating pairs that span a putative inversion. These read-pairs are in FF or RR orientation.
Deletion-pairs: Read-pairs where the two sides represent the breakpoint of a deletion. These read-pairs are in FR relative orientation with the mapping coordinates of both sides is larger than the ‘max_size’ variable, where max_size=(n*MAD of insert size distribution)+(Median of the insert size distribution). ‘n’ is set by the user (default: 7). The program calculates insert size parameters.
Translocation-pairs: Chimeric read-pairs that land on two different chromosomal contigs such that they indicate a read-pair spanning a breakpoint of a translocated structural variant.
Tandem-Duplication-pairs: Read-pairs where the two sides represent one breakpoint of a tandem duplication. These read-pairs are in RF relative orientation with the mapping coordinates of both sides is larger than the ‘max_size’ variable, where max_size=(n*MAD of insert size distribution)+(Median of the insert size distribution). ‘n’ can be set by the user (default: 7). The program calculates insert size parameters.

All reads with unmapped mates can also be marked for further analysis.
All of the categorized read-pairs with both ends mapped in each of the variant types can then be clustered. In some embodiments, the categorized read-pairs with both ends mapped in each of the variant types can be clustered using a hierarchical single-linkage, or nearest-neighbor clustering method using a custom C++ program, readPairCluster v0.1.0, which is described as follows:
   Each read-pair is considered to be part of any specific cluster if both sides of the read-pair fall within a ‘max_dist’ clustering distance variable. max_dist==(n*MAD of insert size distribution)+(Median of the insert size distribution). ‘n’ is set by the user (default: 7). The BamStat program calculates insert size parameters for this variable.
   Clusters are output with an option for restriction to only read-pairs where both sides meet a minimum alignment mapping quality score (default: 0).
   Read-pairs with both sides of 0 mapping quality are removed.
   Each cluster is processed to separate distinct orientation pairs into separate clusters, effectively categorizing multiple derivative breakpoints of a putative structural variant event.

After processing with the categorizing analysis program (e.g., BamStat) and clustering analysis program (e.g., readPairCluster), each output cluster generally represents a putative structural variant breakpoint.

### Module III: Computational Determination of Validity of Structural Variant Breakpoints

The categorized clusters output from Module II can be further analyzed to determine the validity of the clusters of read-pairs representing a true structural variant in the underlying DNA. By way of example only, the categorized clusters output by readPairCluster can be characterized and filtered through a pre-trained decision tree classification program to make decisions on the validity of a cluster of read-pairs representing a true structural variant in the underlying DNA. The inventors have developed a custom program, SVDetect, which employs a random forest classifier to make decisions on the validity of a cluster of read-pairs representing a true structural variant in the underlying DNA.

### Example Algorithms of SVDetect

Read-pair clusters are first filtered if their mapping positions are localized to predefined centromeric, telomeric, or heterochromatic regions over the reference genome assembly.
Each read-pair cluster represents a single putative ‘breakpoint’ of an overall event (e.g. two breakpoints for an inversion represented by two separate clusters, multiple breakpoints for translocations represented by multiple clusters).
At least two or more of the following features can be measured for each cluster:
   a. Cluster Size—the number of read-pairs in the cluster;
   b. Mapping quality scores on both ends of the cluster and the residual between both measurements;
   c. Read-pair uniqueness across both sides of a cluster, or the (number of unique mapping positions/number of total mapping positions) on both ends of a cluster and the residual between both measurements;
   d. ‘Span’ or the distance between the maximum and minimum mapping positions on both ends of a cluster;
      e. Normalized version of the span measurement calibrated to the current library size using measurements from the categorizing program described earlier (e.g., BamStat);
   f. Local Coverage ratio between the number of anomalous pairs to proper pairs at the breakpoint junction in a cluster. Number of anomalous pairs=cluster size, and number of proper pairs is pre-calculated at each base-pair in the genome by SVDetect. This ratio is measured on both sides of a cluster as well as the residual between both measurements;
   g. Global coverage ratio between the number of anomalous pairs to average haploid proper pair coverage in the genome. Average Haploid Coverage=(#Mapped-Proper-Read-Pairs×Median Insert Size)/(Genome Length).
   h. GC percent averaging across sequences of both sides of a cluster, and the residual between both measurements; and
   i. Alignability percent averaging across both sequences of both ends of a cluster and the residual between both measurements.
Each of the features, calculated for all clusters of all variant types, can be fed as input into a pre-trained Random Forest Classifier developed in R programming language, and an output probability assignment is made for each cluster across two classes: VALID and INVALID, representing validity or invalidity of a cluster in describing a true structural variant in the underlying DNA. For example, about 500 trees can be calculated per iteration of the classifier, with four or more decision features used.

### Module IV: Other Optional Analyses

In some embodiments, PCR and/or sequencing (e.g., Sanger sequencing) can be used to validate breakpoint clusters generated by the classifier.

In some embodiments, multiple breakpoints representing distinct ends of an event can be linked together using a specific algorithm to discover both simple and complex rearrangements. For example, use of a custom Python script can allow for the discovery of both simple and complex rearrangements.

In some embodiments, variants can be tagged according to sample, and thus ready for inter-sample or inter-study comparisons and/or for further annotation of disease contribution.

### REFERENCES

1. Update on overall prevalence of major birth defects—Atlanta, Ga. 1978-2005. MMWR Morb Mortal Wkly Rep 2008; 57:1-5.
2. Warburton D. De novo balanced chromosome rearrangements and extra marker chromosomes identified at prenatal diagnosis: clinical significance and distribution of breakpoints. Am J Hum Genet 1991; 49:995-1013.
3. Long G, Sprigg A. A comparative study of routine versus selective fetal anomaly ultrasound scanning J Med Screen 1998; 5:6-10.
4. ACOG Committee Opinion No. 446: array comparative genomic hybridization in prenatal diagnosis. Obstet Gynecol 2009; 114:1161-3.
5. Hochstenbach R, van Binsbergen E, Engelen J, et al. Array analysis and karyotyping: workflow consequences based on a retrospective study of 36,325 patients with idiopathic developmental delay in the Netherlands. Eur J Med Genet 2009; 52:161-9.
6. Korbel J O, Urban A E, Affourtit J P, et al. Paired-end mapping reveals extensive structural variation in the human genome. Science 2007; 318:420-6.
7. Talkowski M E, Ernst C, Heilbut A, et al. Next-generation sequencing strategies enable routine detection of balanced chromosome rearrangements for clinical diagnostics and genetic research. Am J Hum Genet 2011; 88:469-81.
8. Li H, Durbin R. Fast and accurate long-read alignment with Burrows-Wheeler transform. Bioinformatics 2010; 26:58995.
9. Chiang C, Jacobsen J C, Ernst C, et al. Complex reorganization and predominant non-homologous repair following chromosomal breakage in karyotypically balanced germline rearrangements and transgenic integration. Nat Genet 2012; 44:390-7.
10. Talkowski M E, Mullegama S V, Rosenfeld J A, et al. Assessment of 2q23.1 microdeletion syndrome implicates MBD5 as a single causal locus of intellectual disability, epilepsy, and autism spectrum disorder. Am J Hum Genet 2011; 89:551-63.
11. Talkowski M E, Rosenfeld J A, Blumenthal I, et al. Sequencing chromosomal abnormalities reveals neurodevelopmental loci that confer risk across diagnostic boundaries. Cell 2012; 149:525-37.
12. Janssen N, Bergman J E, Swertz M A, et al. Mutation update on the CHD7 gene involved in CHARGE syndrome. Hum Mutat 2012; 33:1149-60.
13. Rutsch F, Gailus S, Miousse I R, et al. Identification of a putative lysosomal cobalamin exporter altered in the cblF defect of vitamin B12 metabolism. Nat Genet 2009; 41:234-9.
14. Blake K D, Davenport S L, Hall B D, et al. CHARGE association: an update and review for the primary pediatrician. Clin Pediatr (Phila) 1998; 37:159-73.
15. Pagon R A, Graham J M Jr, Zonana J, Yong S L. Coloboma, congenital heart disease, and choanal atresia with multiple anomalies: CHARGE association. J Pediatr 1981; 99:223-7.
16. Schnetz M P, Bartels C F, Shastri K, et al. Genomic distribution of CHD7 on chromatin tracks H3K4 methylation patterns. Genome Res 2009; 19:590-601.
17. Park H, Kim J I, Ju Y S, et al. Discovery of common Asian copy number variants using integrated high-resolution array CGH and massively parallel DNA sequencing. Nat Genet 2010; 42:400-5.
18. Shaikh T H, Gai X, Perin J C, et al. High-resolution mapping and analysis of copy number variations in the human genome: a data resource for clinical and research applications. Genome Res 2009; 19: 1682-90.
19. Hara Y, Hirota K, Fukuda K. Successful airway management with use of a laryngeal mask airway in a patient with CHARGE syndrome. J Anesth 2009; 23:630-2.

1. A method of prenatal determination of chromosomal abnormalities comprising:
a. subjecting genomic DNA extracted from cells in an amniotic fluid sample to whole-genome sequence analysis using a large-insert jumping library; and
b. identifying, using a specifically-programmed computer system, structural rearrangements or chromosomal breakpoints in the DNA based on the sequencing data, thereby detecting the presence of one or more abnormalities in the genomic DNA of a fetus associated with structural rearrangements or chromosomal breakpoints.

2. A method of postnatal determination of chromosomal abnormalities comprising:
a. subjecting genomic DNA extracted from tissue to whole-genome sequence analysis using a large-insert jumping library; and
b. identifying, using a specifically-programmed computer system, structural rearrangements or chromosomal breakpoints in the DNA based on the sequencing data, thereby detecting the presence of one or more abnormalities in the genomic DNA of a human subject associated with structural rearrangements or chromosomal breakpoints.

3. The method of claim 1, wherein the large-insert jumping library is created by a process comprising:
a. size-selecting fragments of the genomic DNA;
b. circularizing the size-selected DNA fragments with adaptors comprising a first member of an affinity binding pair, and an optional endonuclease recognition site;
c. fragmenting the circularized DNA into linear DNA fragments in the presence of an endonuclease specific for the endonuclease recognition site present in the adaptors or by random shearing the circularized DNA, wherein at least a portion of the linear DNA fragments comprise the adaptors of step (b) and an end sequence derived from the genomic DNA on each end of the linear DNA fragments;
d. contacting the linear DNA fragments with a solid support comprising a second member of the affinity binding pair, thereby selecting linear DNA fragments comprising the first member of the affinity binding pair and the end sequence on either end;
e. amplifying the linear DNA fragments bound to the solid support, thereby generating a library of DNA fragments comprising end sequences derived from the genomic DNA, wherein the end sequences are separated by a genomic distance equal to the size of the size-selected DNA fragments.

4. The method of claim 3, wherein the size-selected DNA fragments are approximately 2 kb to 6 kb.

5. The method of claim 3, wherein the optional endonuclease recognition site is EcoP15I restriction site.

6. The method of claim 3, wherein the first member of the affinity binding pair comprises a biotinylated nucleotide.

7. The method of claim 6, wherein the second member of the affinity binding pair comprises streptavidin.

8. The method claim 3, wherein the end sequence on either end has a length of about 50 bp to about 200 bp of the genomic sequence.

9. The method of claim 3, wherein the amplifying of step (e) is performed by polymerase chain reaction.

10. The method of claim 3, wherein the oligonucleotide barcode is sample-specific, thereby allowing simultaneous amplification of more than one sample in the same amplification reaction.

11. The method of claim 1, wherein the specifically-programmed computer system comprises one or more processors; and memory to store one or more programs, the one or more programs comprising instructions for:
a. aligning paired-end sequence reads obtained from the whole-genome sequence analysis against sequence of at least one or more chromosomes;
b. categorizing as anomalous, those read pairs that align to genomic sequences separated by significantly greater than or less than the size of the DNA fragments selected for library creation, that have unexpected orientations, or for which the corresponding end sequences align to different chromosomes;
c. categorizing the anomalous read pairs into the same cluster if both sides of the read pairs align within a selected distance of each other; wherein each output cluster represents a putative structural variant breakpoint; and
d. displaying a content that comprises a signal indicative of information associated with the output clusters, wherein the signal is selected from the group consisting of a signal indicative of one or more detectable structural variant breakpoints; a signal indicative of no detectable structural variant breakpoints; a signal indicative of a normal sample, a signal indicative of a disease or disorder associated with the detectable structural variant breakpoints, and any combination thereof.

12. The method of claim 11, wherein the structural variant breakpoints are induced by structural rearrangements selected from the group consisting of inversion, deletion, translocation, excision, insertion, duplicated-insertion, tandem-duplication, and any combinations thereof.

13. The method of claim 1, wherein the sequencing is performed on an Illumina™ platform.

14. A system comprising
a. a determination module configured to receive said at least one test sample and perform at least one sequencing analysis on said at least one test sample;
b. a storage device configured to store output sequence data from said determination module;
c. a computing module comprising specifically-programmed instructions to determine from the output sequence data validity or invalidity of structural rearrangements represented by the output sequence data, wherein the instructions comprise:
mapping read-pairs of the output sequence data against a reference genome;
categorizing the read-pairs into clusters based on at least one common feature;
removing the read-pair clusters having their mapping positions localized to predefined centromeric, telomeric, or heterochromatic regions over the reference genome;
measuring at least two or more features of the remaining read-pair clusters, wherein said features are selected from the group consisting of:
i. number of read-pairs in the cluster;
ii. mapping quality scores on both ends of the cluster and the residual between both measurements;
iii. read-pair uniqueness across both ends of the cluster and the residual between both measurements;
iv. distance between the maximum and minimum mapping positions on both ends of the cluster;
v. normalized distance between the maximum and minimum mapping positions on both ends of the cluster;
vi. local coverage ratio of the number of the read-pairs in the cluster to the number of proper pairs at a breakpoint junction in the cluster;
vii. global coverage ratio of the number of the read-pairs in the cluster to average haploid proper pair coverage in the reference genome;
viii. GC percent averaging across regions on both ends of the cluster and the residual between both measurements;
ix. alignability percent averaging across sequences of both ends of the cluster and the residual between both measurements; and
x. any combinations thereof;
performing a pre-trained decision tree classification program to determine the validity or invalidity of the structural rearrangements represented by the clusters; and
d. a display module for displaying a content based in part on the data output from said computing module, wherein the content comprises a signal indicative of the presence of valid structural rearrangements, or a signal indicative of the absence of any valid structural rearrangements.

15. The system of claim 14, wherein the sequencing analysis is based on a large-insert jumping library.
